(19) United States
(12) Patent Application Publication
(10) Pub. No.: US 2007/0067034 A1
(43) Pub. Date: Mar. 22, 2007

(54) IMPLANTABLE DEVICES AND METHODS FOR TREATING MICRO-ARCHITECTURE DETERIORATION OF BONE TISSUE

(76) Inventors: Paul E. Chirico, Campbell, CA (US); Ben M. Chan, Fremont, CA (US)

Correspondence Address: WILSON SONSINI GOODRICH & ROSATI, 650 PAGE MILL ROAD, PALO ALTO, CA, 94304-1050, US

(21) Appl. No.: 11/468,759

(22) Filed: Aug. 30, 2006

Related U.S. Application Data
(60) Provisional application No. 60/713,259, filed on Aug. 31, 2005.

(51) Int. Cl. A61F 2/44; A61B 17/16

(52) U.S. Cl. 623/17.11; 606/79

(57) ABSTRACT

An expandable stabilization device is disclosed that is suitable for deployment within cancellous bone, including, for example, within a vertebral body of a spine. The device comprises: an elongate expandable shaft adapted to be positioned within a vertebral body having a first profile and a second profile; wherein the shaft is adapted to cut through cancellous bone within the vertebral body during expansion from the first profile to the second profile; and further wherein the shaft is adapted to abut a surface of cortical bone within the vertebral body without passing therethrough. The invention also includes a method for treating cancellous bone, such as cancellous bone of a vertebral body. The method comprises: delivering an expandable device within the cancellous bone of in an interior of a vertebral body; expanding the delivered device within the cancellous bone of the vertebra body; applying force from a surface of the device to an inner surface of a cancellous bone of the vertebral body sufficient to cut through the cancellous bone; and applying force from a surface of the device to an inner surface of a cortical bone of the vertebral body sufficient to support the vertebral body

### CROSS-REFERENCE

This application claims the benefit of U.S. Provisional Application No. 60/713,259, filed Aug. 31, 2005, entitled Implantable Device for Treating VCF, Tools and Methods which is incorporated herein by reference in its entirety.

### BACKGROUND OF THE INVENTION

1. Field of the Invention

The invention relates to devices, implants and methods for treating and supporting cancellous bone within a skeletal structure. The invention also relates to devices, implants and methods for treating and supporting cancellous bone within vertebral bodies, particularly vertebral bodies which have suffered a vertebral compression fracture (VCF).

2. Description of the Related Art

Micro-architecture deterioration of bone tissue can result from a variety of factors including, disease, aging, stress and use. One such example is osteoporosis, which is a disease characterized by low bone mass and micro-architecture deterioration of bone tissue. Osteoporosis leads to bone fragility and an increase fracture risk. The World Health Organization defines osteoporosis as a bone density more than 2.5 standard deviations below the young adult mean value. Values between 1 and 2.5 standard deviation below the young adult mean are referred to as osteopenia.

While osteoporosis affects the entire skeleton, it most commonly causes fractures in the spine and hip. As can easily be appreciated, spinal or vertebral fractures have serious consequences, with patients suffering from loss of height, deformity and persistent pain which can significantly impair mobility and quality of life. An estimated 1.5 million elderly people in the United States suffer an osteoporotic fracture each year. Of these fractures, an estimated 750,000 are vertebral compression fractures (VCFs) and 250,000 are hip fractures. VCFs in women age 50 and older is estimated to be greater than 25%, with the rate increasing with age. Fracture pain usually lasts 4 to 6 weeks, with intense pain at the fracture site.

In an osteoporotic bone, pores or voids in the sponge-like cancellous bone increase in dimension, making the bone very fragile. Although in young, healthy bone tissue, bone breakdown occurs continually as the result of osteoclast activity, the breakdown is balanced by new bone formation by osteoblasts. In contrast, in an elderly patient, bone resorption can surpass bone formation thus resulting in deterioration of bone density. Osteoporosis occurs largely without symptoms until a fracture occurs.

While there have been pharmaceutical advances aimed toward slowing or arresting bone loss, new and improved solutions to treating VCFs are still needed in view of the expectancy that the number of people suffering from VCFs will grow steadily as life expectancy increases.

As illustrated in FIG. 1, the spine is comprised of a plurality of vertebral bodies with intervening intervertebral discs. Both the width and depth of the vertebral bodies increase as the spine descends in the rostral-to-caudal direction. Additionally the height of the vertebral bodies also increase in the rostral-to-caudal direction, with the exception of a slight reversal at C6 and lower lumbar levels.

Vertebral bodies, as well as other skeletal bones, are made up of a thick cortical shell and an inner meshwork of porous cancellous bone. Cancellous bone is comprised of collagen, calcium salts and other minerals. Cancellous bone also has blood vessels and bone marrow in the spaces.

Vertebroplasty and kyphoplasty are recently developed techniques for treating vertebral compression fractures. Percutaneous vertebroplasty was first reported by in 1987 for the treatment of hemangiomas. In the 1990's, percutaneous vertebroplasty was extended to indications including osteoporotic vertebral compression fractures, traumatic compression fractures, as well as vertebral metastasis. In one percutaneous vertebroplasty technique, bone cement such as PMMA (polymethylmethacrylate) is percutaneously injected into a fractured vertebral body through a trocar and cannula system. The targeted vertebrae are identified under fluoroscopy. A needle is introduced into the vertebral body under fluoroscopic control to allow direct visualization. A transpedicular (through the pedicle of the vertebrae) approach is typically bilateral but can be done unilaterally. The bilateral transpedicular approach is typically used because inadequate PMMA infill is achieved with a unilateral approach.

In a bilateral approach, approximately 1 to 4 ml of PMMA are injected on each side of the vertebra. Since the PMMA needs to be forced into cancellous bone, the technique requires high pressures and fairly low viscosity cement. Since the cortical bone of the targeted vertebra may have a recent fracture, there is the potential of PMMA leakage. The PMMA cement contains radiopaque materials so that when injected under live fluoroscopy, cement localization and leakage can be observed. The visualization of PMMA injection and extravasion are critical to the technique and the physician terminates PMMA injection when leakage is evident. The cement is injected using small syringe-like injectors to allow the physician to manually control the injection pressures.

Kyphoplasty is a modification of percutaneous vertebroplasty. Kyphoplasty involves a preliminary step that comprises the percutaneous placement of an inflatable balloon tamp in the vertebral body. Inflation of the balloon creates a cavity in the bone prior to cement injection. Further, the proponents of percutaneous kyphoplasty have suggested that high pressure balloon-tamp inflation can at least partially restore vertebral body height. In kyphoplasty, it has been proposed that PMMA can be injected at lower pressures into the collapsed vertebra since a cavity exists within the vertebral body to receive the cement—which is not the case in conventional vertebroplasty.

The principal indications for any form of vertebroplasty are osteoporotic vertebral collapse with debilitating pain. Radiography and computed tomography must be performed in the days preceding treatment to determine the extent of vertebral collapse, the presence of epidural or foraminal stenosis caused by bone fragment retropulsion, the presence of cortical destruction or fracture and the visibility and degree of involvement of the pedicles. Leakage of PMMA during vertebroplasty can result in very serious complications including compression of adjacent structures that necessitate emergency decompressive surgery.

The human spinal column 10, as shown in FIG. 1A, is comprised of a series of thirty-three stacked vertebrae 12 divided into five regions. The cervical region includes seven vertebrae, known as C1-C7. The thoracic region includes twelve vertebrae, known as T1-T12. The lumbar region contains five vertebrae, known as L1-L5. The sacral region is comprised of five fused vertebrae, known as S1-S5, while the coccygeal region contains four fused vertebrae, known as Co1-Co4.

An example of one vertebra is illustrated in FIG. 1B which depicts a superior plan view of a normal human lumbar vertebra 12. Although human lumbar vertebrae vary somewhat according to location, the vertebrae share many common features. Each vertebra 12 includes a vertebral body 14. Two short boney protrusions, the pedicles, extend dorsally from each side of the vertebral body 14 to form a vertebral arch 18 which defines the vertebral foramen.

At the posterior end of each pedicle, the vertebral arch 18 flares out into broad plates of bone known as the laminae 20. The laminae 20 fuse with each other to form a spinous process 22. The spinous process 22 provides for muscle and ligamentous attachment. A smooth transition from the pedicles to the laminae 20 is interrupted by the formation of a series of processes. Two transverse processes thrust out laterally, one on each side, from the junction of the pedicle with the lamina 20. The transverse processes serve as levers for the attachment of muscles to the vertebrae 12. Four articular processes, two superior and two inferior, also rise from the junctions of the pedicles and the laminae 20. The superior articular processes are sharp oval plates of bone rising upward on each side of the vertebrae, while the inferior processes 28, 28′ are oval plates of bone that jut downward on each side.

The superior and inferior articular processes each have a natural bony structure known as a facet. The superior articular facet faces medially upward, while the inferior articular facet faces laterally downward. When adjacent vertebrae 12 are aligned, the facets, capped with a smooth articular cartilage and encapsulated by ligaments, interlock to form a facet joint 32. The facet joints are apophyseal joints that have a loose capsule and a synovial lining.

An intervertebral disc 34 between each adjacent vertebra 12 (with stacked vertebral bodies shown as 14, 15 in FIG. 1C) permits gliding movement between the vertebrae 12. The structure and alignment of the vertebrae 12 thus permit a range of movement of the vertebrae 12 relative to each other. FIG. 1D illustrates a posterolateral oblique view of a vertebrae 12. The vertebral body 14 is shown in a cut-away that illustrates the cortical bone 40 which forms the exterior of the bone (in this case the vertebral body) and the spongy cancellous bone 42 located within the interior of the cortical bone.

Despite the small differences in mineralization, the chemical composition and true density of cancellous bone are similar to those of cortical bone. As a result, the classification of bone tissue as either cortical or cancellous is based on bone porosity, which is the proportion of the volume of bone occupied by non-mineralized tissue. Cortical bone has a porosity of approximately 5-30% whereas cancellous bone porosity may range from approximately 30 to more than 90%. Although typically cortical bone has a higher density than cancellous bone, that is not necessarily true in all cases. As a result, for example, the distinction between very porous cortical bone and very dense cancellous bone can be somewhat arbitrary.

The mechanical strength of cancellous bone is well known to depend on its apparent density and the mechanical properties have been described as those similar to man-made foams. Cancellous bone is ordinarily considered as a two-phase composite of bone marrow and hard tissue. The hard tissue is often described as being made of trabecular “plates and rods.” Cancellous microstructure can be considered as a foam or cellular solid since the solid fraction of cancellous bone is often less than 20% of its total volume and the remainder of the tissue (marrow) is ordinarily not significantly load carrying. The experimental mechanical properties of trabecular tissue samples are similar to those of many man-made foams. If a sample of tissue is crushed under a prescribed displacement protocol, the load-displacement curve will initially be linear, followed by an abrupt nonlinear “collapse” where the load carrying capacity of the tissue is reduced by damage. Next follows a period of consolidation of the tissue where the load stays essentially constant, terminated by a rapid increase in the load as the tissue is compressed to the point where the void space is eliminated. Each of the mechanical properties of cancellous bone varies from site-to-site in the body. The apparent properties of cancellous bone as a structure depend upon the conformation of the holes and the mechanical properties of the underlying hard tissue composing the trabeculae. The experimental observation is that the mechanical properties of bone specimens are power functions of the solid volume fraction. The microstructural measures used to characterize cancellous bone are very highly correlated to the solid volume fraction. This suggests that the microstructure of the tissue is a single parameter function of solid volume fraction. If this is true, the hard tissue mechanical properties will play a large role in determining the apparent properties of the tissue. At this time, little is known about the dependence of trabecular hard tissue mechanical properties on biochemical composition or ultrastructural organization.

Cancellous bone in the joints and spine is continuously subject to significant loading. One consequence of this is that the tissue can experience, and occasionally accumulate, microscopic fractures and cracks. These small damages are similar to those seen in man-made materials and are, in many cases, the result of shear failure of the material. It is known that microcracks accumulate with age in the femoral head and neck, leading to a hypothesis that these damages are related to the increase in hip fracture with age. However, no such association of increased crack density with age was found in human vertebral cancellous bone despite the high incidence of spinal fractures, particularly in women.

Adult cortical and cancellous bone can be considered as a single material whose apparent density varies over a wide range. The compressive strength of bone tissue is proportional to the square of the apparent density.

Cortical bone morphology and composition can be characterized by an examination of microstructure, porosity, mineralization, and bone matrix. These parameters seldom vary independently but are usually observed to vary simultaneously. Mechanical properties vary through the cortical thickness due to variations in microstructure, porosity, and chemical composition.

Mechanical properties are dependent on microstructure. The strongest bone type is circumferential lamellar bone, followed in descending order of strength by primary laminar, secondary Haversian, and woven-fibered bone. All normal adult cortical bone is lamellar bone. Most of the cortical thickness is composed of secondary Haversian bone. Circumferential lamellar bone is usually present at the endosteal and periosteal surfaces. In the adult, woven-fibered bone is formed only during rapid bone accretion, which accompanies conditions such as fracture callus formation, hyperparathyroidism, and Paget's disease.

Aging is associated with changes in bone microstructure which are caused primarily by internal remodeling throughout life. In the elderly, the bone tissue near the periosteal surface is stronger and stiffer than that near the endosteal surface due primarily to the porosity distribution through the cortical thickness caused by bone resorption. Bone collagen intermolecular cross-linking and mineralization increase markedly from birth to 17 years of age and continue to increase, gradually, throughout life. Adult cortical bone is stronger and stiffer and exhibits less deformation to failure than bone from children. Cortical bone strength and stiffness are greatest between 20 and 39 years of age. Further aging is associated with a decrease in strength, stiffness, deformation to failure, and energy absorption capacity

From this understanding of bone, it can be appreciated that when a vertebral body becomes damaged, as illustrated in FIG. 1E, such as when a fracture 80 occurs, a portion of the vertebral body typically collapses. This collapse can occur as a result of micro-architecture deterioration of the bone tissue.

The terms caudal and cephalad may be used in conjunction with the devices and operation of the devices and tools herein to assist in understanding the operation and/or position of the device and/or tools.

In order to understand the configurability, adaptability, and operational aspects of the invention disclosed herein, it is helpful to understand the anatomical references of the body 50 with respect to which the position and operation of the devices, and components thereof, are described. There are three anatomical planes generally used in anatomy to describe the human body and structure within the human body: the axial plane 52, the sagittal plane 54 and the coronal plane 56 (see FIG. 1F). Additionally, devices and the operation of devices and tools are better understood with respect to the caudad 60 direction and/or the cephalad direction 62. Devices and tools can be positioned dorsally 70 (or posteriorly) such that the placement or operation of the device is toward the back or rear of the body. Alternatively, devices can be positioned ventrally 72 (or anteriorly) such that the placement or operation of the device is toward the front of the body. Various embodiments of the devices, systems and tools of the present invention may be configurable and variable with respect to a single anatomical plane or with respect to two or more anatomical planes. For example, a component may be described as lying within and having adaptability or operability in relation to a single plane. For example, a device may be positioned in a desired location relative to an axial plane and may be moveable between a number of adaptable positions or within a range of positions. Similarly, the various components can incorporate differing sizes and/or shapes in order to accommodate differing patient sizes and/or anticipated loads.

### SUMMARY OF THE INVENTION

In an embodiment of the invention, an expandable stabilization device for deployment within a vertebral body of a spine is provided. The device comprises: an elongate expandable shaft adapted to be positioned within a vertebral body having a first profile and a second profile; wherein the shaft is adapted to cut through cancellous bone within the vertebral body during expansion from the first profile to the second profile; and further wherein the shaft is adapted to abut a surface of cortical bone within the vertebral body without passing therethrough.

In another embodiment of the invention, an expandable stabilization device for deployment within a target section of cancellous bone is provided. The device comprises: an elongate expandable shaft adapted to be positioned within a cancellous bone having a first profile and a second profile; wherein the shaft is adapted to cut through cancellous bone during expansion from the first profile to the second profile; and further wherein the shaft is adapted to abut a surface of cortical bone surrounding the cancellous bone without passing therethrough.

In yet another embodiment of the invention, a system is provided for cutting through cancellous bone without cutting through cortical bone. The system comprises: an expandable body having a first profile and a second profile wherein a surface of the expandable body is adapted to cut through cancellous bone; and a delivery device having a distal end adapted to engage the expandable body to deliver the delivery device into the cancellous bone of a body.

In still another embodiment, an expandable device is provided that is adapted to apply force sufficient to cut through cancellous bone and insufficient to cut through a cortical bone section during expansion of the device wherein the device restores a height of a vertebral body to a target height.

In another embodiment, a cannula is provided that is adapted to be deployed within cancellous bone, such as cancellous bone in a vertebral body of a spine comprising: an elongate expandable tube adapted to be positioned within cancellous bone having a first profile and a second profile; wherein the tube is adapted to cut through cancellous bone during expansion from the first profile to the second profile; further wherein the tube is adapted to deliver a target material through the elongate expandable tube into the cancellous bone; and further wherein the tube is adapted to abut an interior cortical bone surface without completely passing therethrough.

In yet another embodiment, an expandable device for use in treating a fractured or collapsed bone, such as a fractured or collapsed vertebral body of a spine, is provided. The device comprises: a device adapted to cut through cancellous bone interior the bone and abut an inner surface of cortical bone comprising an elongate expandable shaft adapted to be positioned with the bone having a delivery profile and a deployed profile; and wherein the device selectively expands along its length in the deployed profile to selectively restore the height of a portion of the fractured or collapsed bone to a target dimension.

In still another embodiment, a system for cutting through cancellous bone, such as the cancellous bone of a vertebral body of a spine, is provided. The system comprises an expandable body having a selectively expandable surface adapted to expand in situ in an angled direction non-parallel to a sagittal plane of the bone and non-parallel to a transverse plane of the bone.

In still another embodiment, a stabilization device for deployment within a bone, such as a vertebral body of a spine, is provided. The stabilization device comprises: an elongate expandable shaft having a first profile and a second profile; a cutting surface on at least a portion of the expandable shaft; wherein the cutting surface cuts through cancellous bone; and further wherein the cutting surface abuts a surface of cortical bone within the bone without passing therethrough.

With any of the embodiments of the device, further embodiments can provide that the elongate shaft comprises a plurality of surface areas at least a portion of which is a cutting surface adapted to apply a cortical bone cutting force to the cortical bone of the vertebral body. The cancellous bone cutting surfaces can be adapted to deliver a force sufficient to cut through the cancellous bone. Suitable forces can be as low as 2 psi to over 100 psi. Sizes of the devices and components can vary depending upon the anatomy to be treated. Dimensions for an an undeployed device typically has a diameter of from 2 mm to 10 mm; a deployed device has a diameter of from 6 mm to 35 mm along at least a portion of its length; and devices typically have a length of from 8 mm to 60 mm.

In still other embodiments of any of the devices, the elongate shaft can be configured to have 2 or more elongate slits along its length. Notches can be provided symmetrically or asymmetrically along the length of the slit. Additionally, the slits can be tapered, as well as symmetrical or asymmetrically positioned on the shaft. The elongate shaft may be self-expanding, or may be controllably expandable. Once expanded, the shaft typically is adapted to support a compressive load and expands to a profile sufficient to achieve a target distance between two cortical bone surfaces, such as a target vertebral body height. In some embodiments, the shaft is adapted to expand more in a first direction than in a second direction; in other embodiments, the shaft expands equally in all directions. In other embodiments, the shaft has a circular cross-section; in other embodiments, the shaft has an oval cross-section. In still further embodiments of any of the devices, the elongate shaft has a first section that is expandable to a first profile and a second section expandable to a second profile.

In still another embodiment of any of the devices, the elongate shaft has a pair of open ended slits at an end of the shaft.

In yet other embodiments of any of the devices, a delivery device is provided that is adapted to establish a subcutaneous path into the target bone.

In still another embodiment, of any of the devices a control member. The control member can be positioned within a lumen of the shaft configured to expand the shaft from the first profile to the second profile. Additionally, the device can further comprise a cannula with a lumen through which material is delivered into the bone. In any of the embodiments, all or part of the device, can be made of any suitable biocompatible material or shape memory material. Additionally, all or part of the surface of the device can be modified to prevent slippage or movement, such as by providing dimples, nubs, knurls or teeth.

In yet another embodiment, a method for treating cancellous bone is provided. The method comprises: delivering an expandable device within the cancellous bone; expanding the delivered device within the cancellous bone; applying force from a surface of the device to an inner surface of a cancellous bone sufficient to cut through the cancellous bone; and applying force from a surface of the device to an inner surface of a cortical bone sufficient to support the cortical bone. In some embodiments, the method can further comprise the step of applying force from the surface of the device to the cortical bone of a vertebral body sufficient to increase the distance between two opposing cortical bone surfaces. In other embodiments, the method can further comprise the step of confirming a position of a vertebral body. In still other embodiments, the method can comprise the step of administering a material within the cortical bone to facilitate bone restoration. In yet other embodiments, the method can comprise the step of administering a material within the cortical bone to stabilize a position of the device within the vertebral body. In still other embodiments, the method further comprises the step of applying force from the surface of the device to the cortical bone sufficient to increase a distance between a first section of the cortical bone and a second section of the vertebral body at a target location within the bone and/or applying force from the surface of the device to the cortical bone sufficient to increase a distance between a caudad cortical section of a vertebral body and a cephalad cortical section of a vertebral body.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a lateral view of a normal human spinal column; FIG. 1B is a superior view of a normal human lumbar vertebra; FIG. 1C is a lateral view of a functional spinal unit having two vertebral bodies and an intervertebral disc; FIG. 1D is a posterolateral oblique view of a vertebrae; FIG. 1E illustrates a portion of a spine wherein a vertebral body is fractured; FIG. 1F illustrates a human body with the planes of the body identified;

FIGS. 2A-S illustrate an embodiment of the invention; FIG. 2A illustrates a perspective view of the device; FIG. 2B illustrates a lateral view of the device; FIGS. 2C-E illustrate cross-sectional views of the device taken along the lines C-C, D-D and E-E of FIG. 2B; FIG. 2F is a cross-sectional view taken along the lines F-F in FIG. 2C; FIGS. 2G-I are illustrations of the device as it expands from a first a first configuration to a deployed configuration; FIGS. 2J-L are cross-sectional views of the device in a partially deployed condition taken along the planes J, K and L in FIG. 2I; FIG. 2M is a cross-sectional view of the device taken along the plane M of FIG. 2I; FIGS. 2N-S illustrate the device being deployed in a vertebral body of a spine;

FIGS. 3A-P illustrate another embodiment of the invention; FIG. 3A illustrates a perspective view of the device; FIG. 3B illustrates a lateral view of the device; FIGS. 3C-E illustrate cross-sectional views of the device taken along the lines C-C, D-D and E-E of FIG. 3B; FIG. 3F is a cross-sectional view taken along the lines F-F in FIG. 3C; FIGS. 3G-H are illustrations of the device as it expands from a first a first configuration to a deployed configuration; FIGS. 3I-K are cross-sectional views of the device in a partially deployed condition taken along the planes I, J and K in FIG. 3H; FIG. 3L is a cross-sectional view of the device taken along the plane L of FIG. 3H; FIGS. 3M-P illustrate the device being deployed in a vertebral body of a spine;

FIGS. 4A-P illustrate yet another embodiment of the invention; FIG. 4A illustrates a perspective view of the device; FIG. 4B illustrates a lateral view of the device; FIGS. 4C-D illustrate cross-sectional views of the device taken along the lines C-C, and D-D of FIG. 4B; FIG. 4E is a cross-sectional view taken along the lines F-F in FIG. 4C; FIGS. 4F-I are illustrations of the device as it expands from a first a first configuration to a deployed configuration; FIGS. 4J-K are cross-sectional views of the device in a partially deployed condition taken along the planes J and K in FIG. 4I; FIG. 4L is a cross-sectional view of the device taken along the plane L of FIG. 4I; FIGS. 4M-P illustrate the device being deployed in a vertebral body of a spine;

FIGS. 5A-O illustrate yet another embodiment of the invention; FIG. 5A illustrates a perspective view of the device; FIG. 5B illustrates a lateral view of the device in an undeployed condition; FIGS. 5C-E illustrate cross-sectional views of the device taken along the lines C-C, D-D and E-E of FIG. 5B; FIG. 5F is a cross-sectional view taken along the lines F-F in FIG. 5C; FIG. 5G is an illustration of the device in a deployed configuration; FIGS. 5H-J are cross-sectional views of the device in a deployed condition taken along the planes H, I and J in FIG. 5G; FIG. 5K is a cross-sectional view of the device taken along the plane K of FIG. 5G; FIGS. 5L-O illustrate the device being deployed in a vertebral body of a spine;

FIG. 6A illustrates the steps of a method of deploying the device within a vertebral body; FIG. 6B illustrates the steps of a method of removing the device from within a vertebral body.

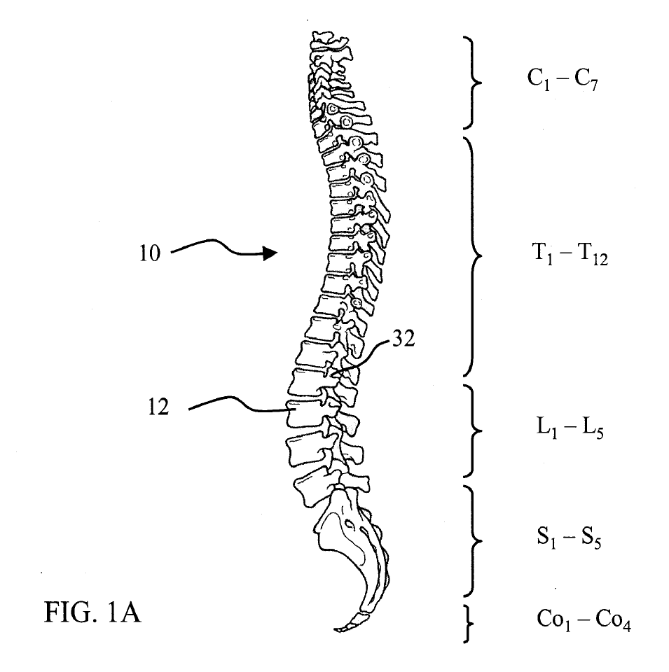

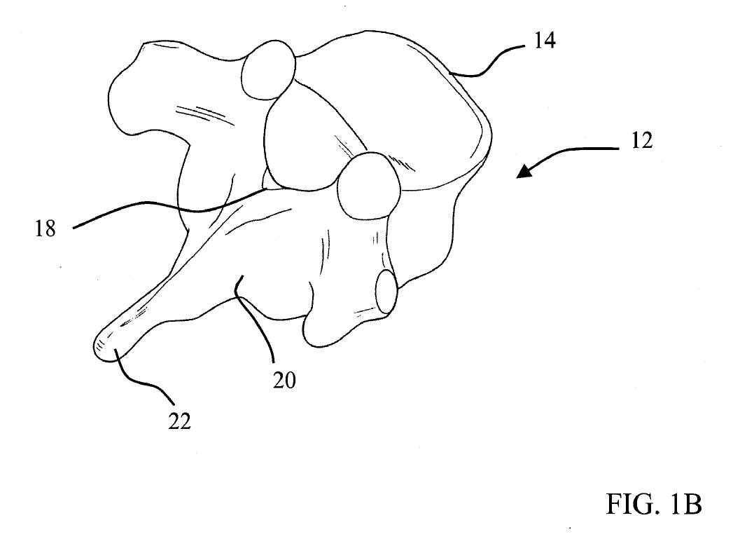

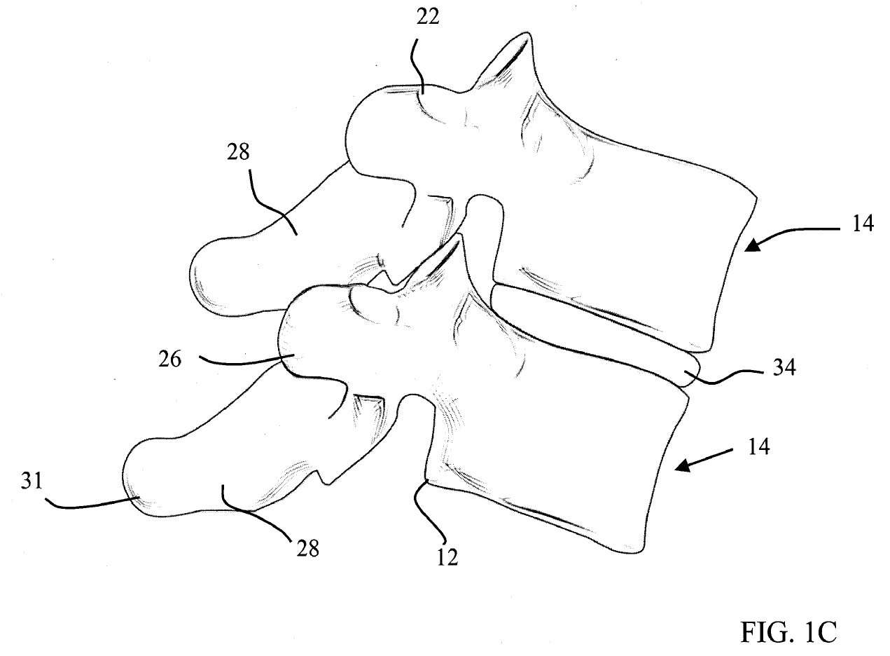

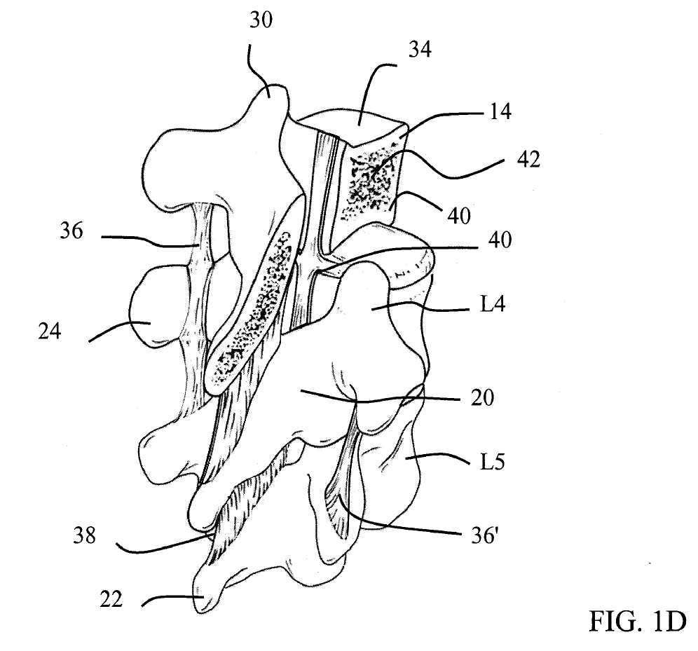

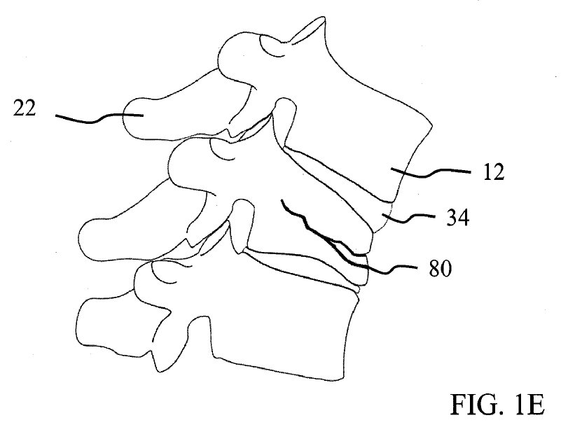

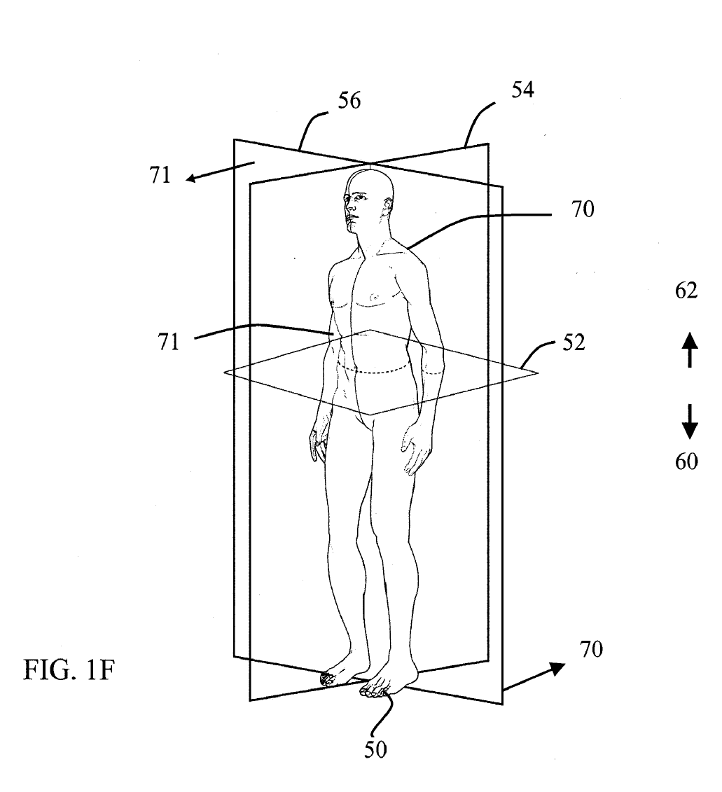

### DETAILED DESCRIPTION OF THE INVENTION

There is a general need to provide systems and methods for use in treatment of fractures and microarchitecture deterioration of bone tissue, such as vertebral compression fractures (“VCFs”), that provides a greater degree of control over introduction of bone support material, and that provide better outcomes. Embodiments of the present invention meet one or more of the above needs, or other needs, and provide several other advantages in a novel and non-obvious manner.

The invention relates to implantable devices and systems suitable for implantation within the body to restore and/or augment connective tissue such as bone, and systems for treating bone and microarchitecture deterioration of bone tissue, including spinal pathologies. The invention relates generally to implantable devices, apparatus or mechanisms that are suitable for implantation within a human body to restore, augment, and/or replace soft tissue and connective tissue, including bone, and systems for treating spinal pathologies. In various embodiments, the implantable devices can include devices designed to replace missing, removed or resected body parts or structure. The implantable devices, apparatus or mechanisms are configured such that the devices can be formed from parts, elements or components which alone, or in combination, comprise the device and systems. The implantable devices can also be configured such that one or more elements or components are formed integrally to achieve a desired physiological, operational or functional result such that the components complete the device. Functional results can include the surgical restoration and functional power of the bone, and/or controlling, limiting or altering the functional power of the bone. Portions of the device can be configured to replace or augment existing anatomy and/or implanted devices, and/or be used in combination with resection or removal of existing anatomical structure. While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

For purposes of illustration, the devices and methods of the invention are described below with reference to the spine. However, as will be appreciation by those skilled in the art, the devices and methods can be employed to address microarchitecture deterioration in any effected bone, including, for example, the hip.

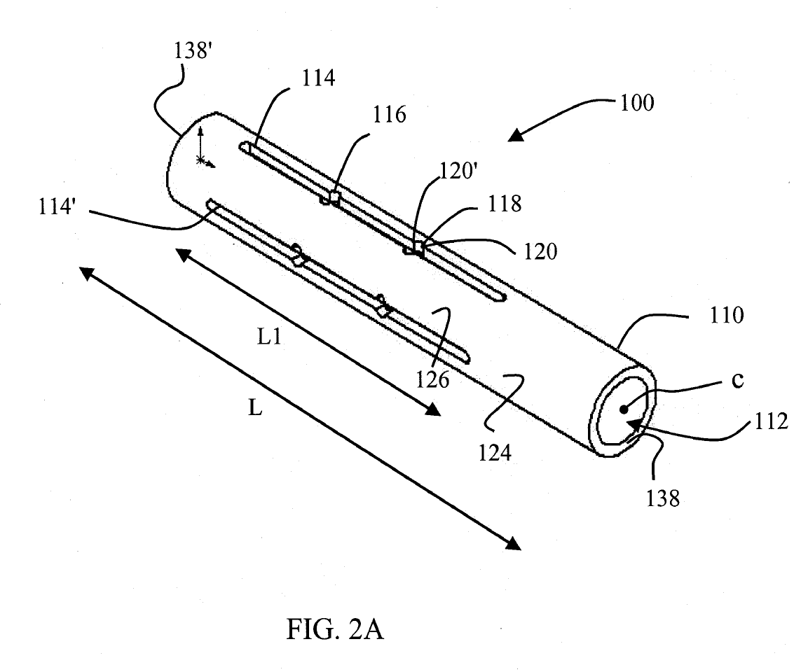

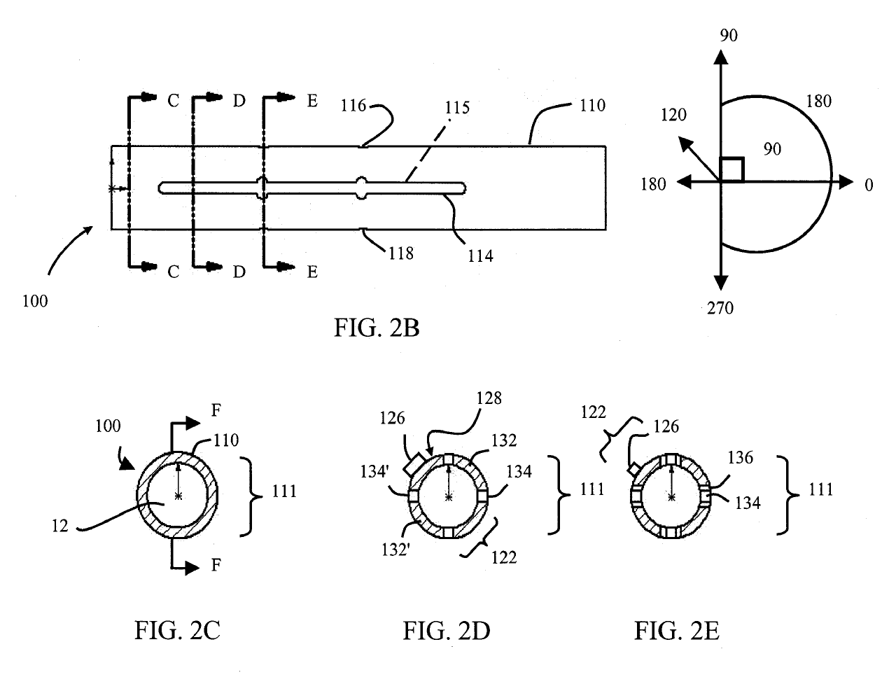

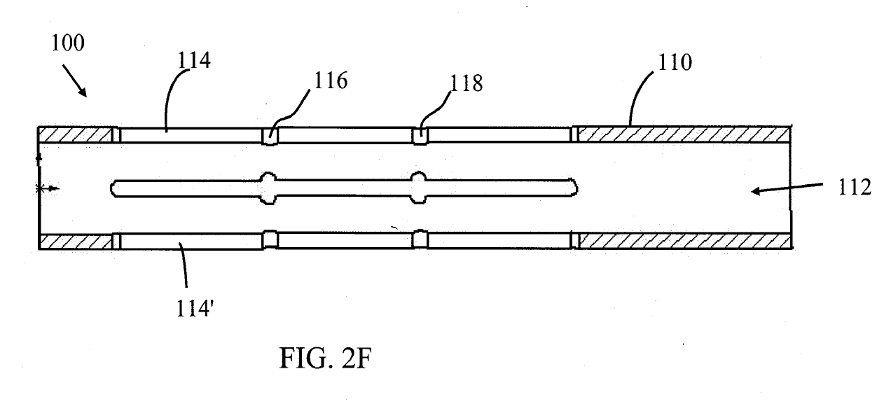

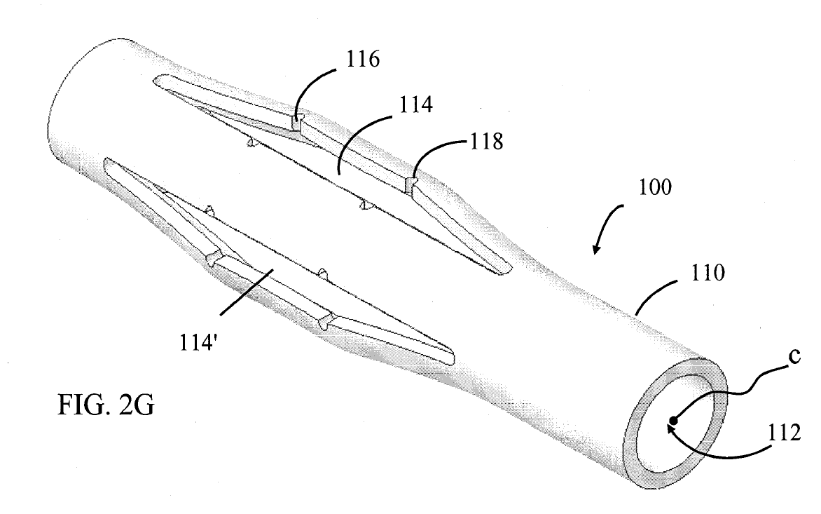

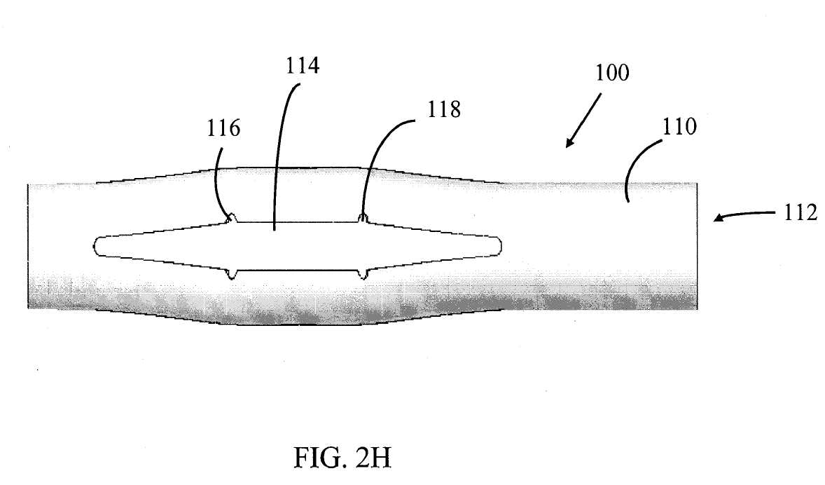

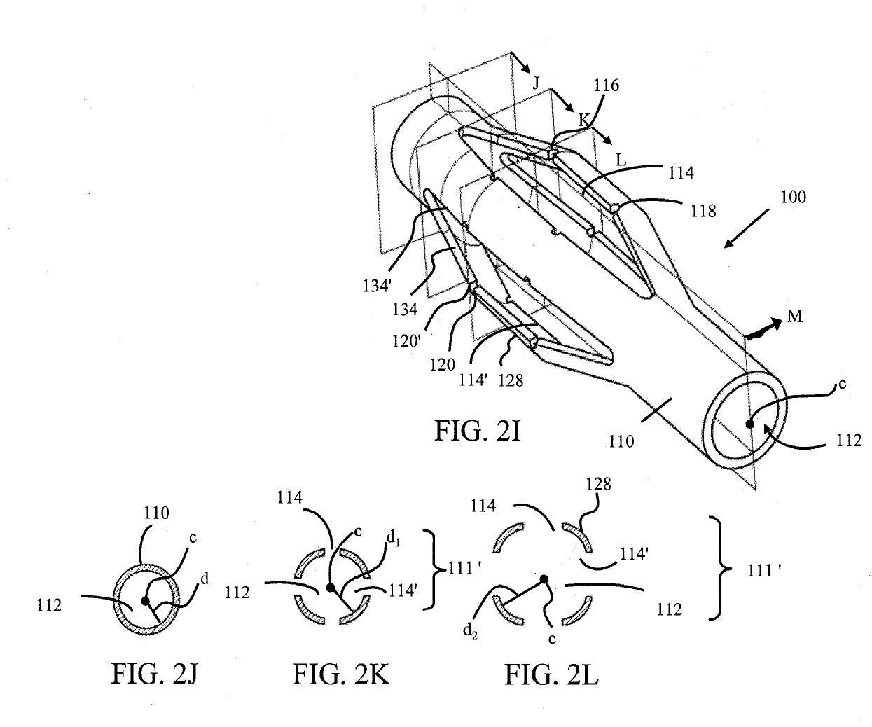

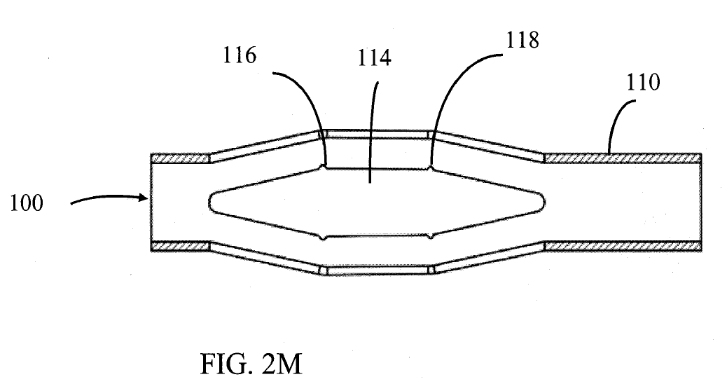

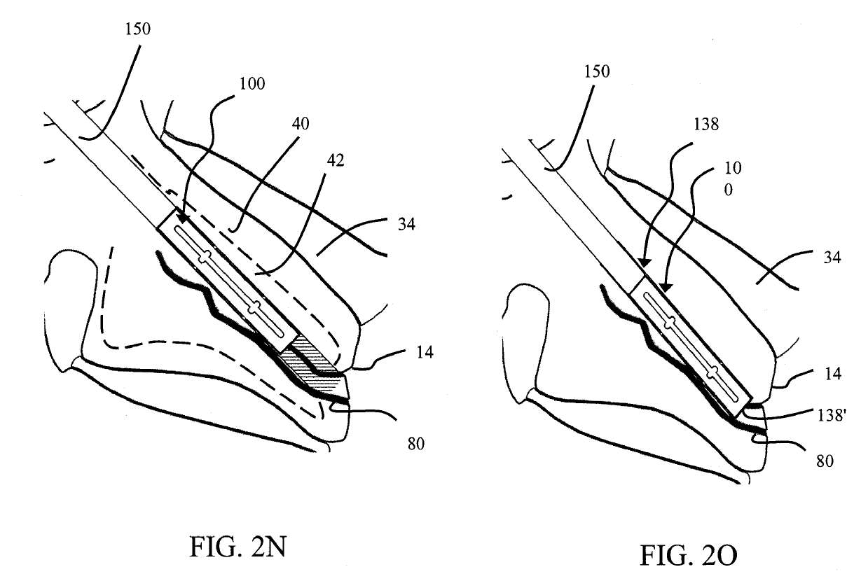

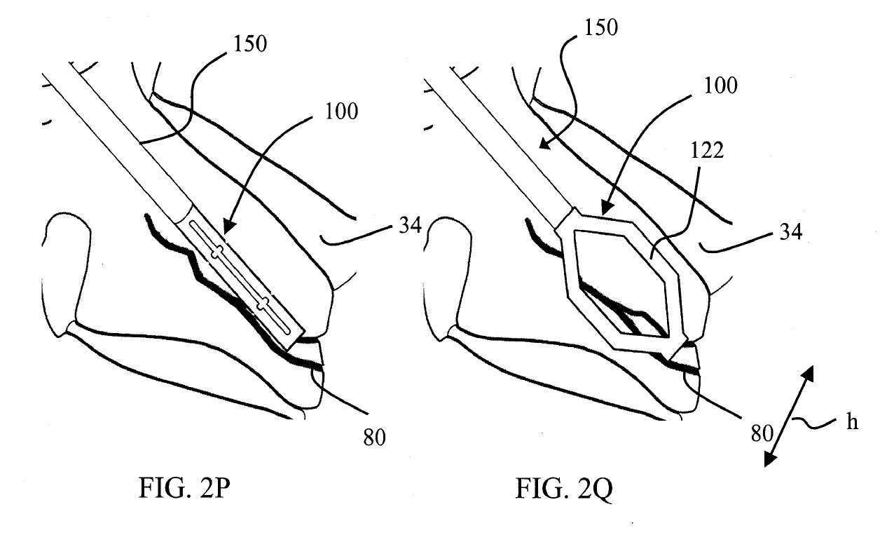

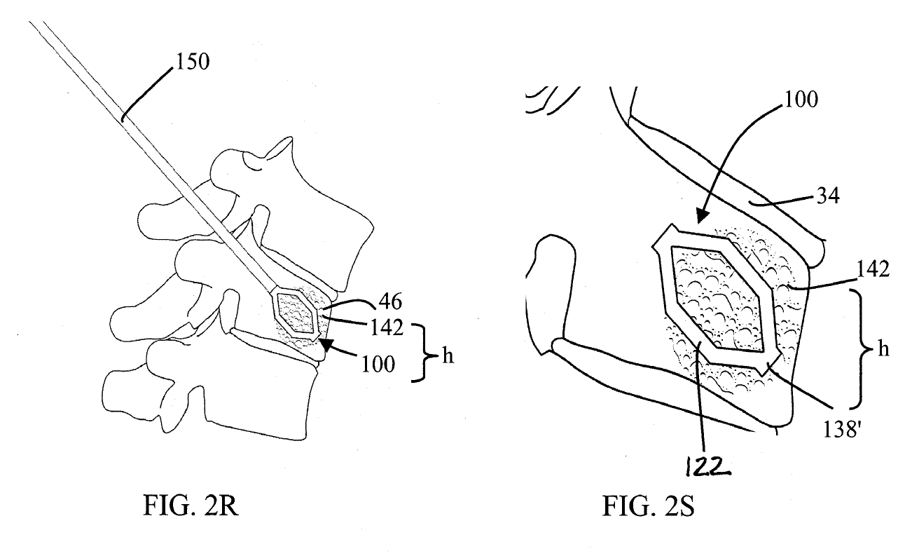

Turning now to a specific embodiment, FIG. 2A illustrates a perspective view of an expandable stabilization device 100 suitable for use completely or partially within a vertebral body 14. The expandable device 100 has an elongate expandable shaft 110 adapted to be positioned within cancellous bone 42, such as the cancellous bone 42 of a vertebral body 14. The non-expandable ends of the device can also be positioned within the cancellous bone, or one end can be partially positioned within cortical bone in order to secure the position of the device.

The elongate expandable shaft 110, as depicted, has a hollow central lumen 112 and two or more slits 114, 114′ along at least a portion L₁ of the length L of the shaft 110. The device, as with all devices disclosed herein, has a proximal end 138, and a distal end 138′. The proximal end is the end closest the user and access point for therapy; the distal end is the end furthest away from the user or delivery device.

Each slit 114 can also be configured to have one or more notches 116, 118 which act as cut-outs in the slits along its length. The device depicted in FIGS. 2B-E is in a first undeployed profile 111. As depicted, the undeployed profile 111 has a constant circumference along its length. However, as will be appreciated by those of skill in the art, the undeployed profile is not restricted to a device having a constant profile along its length and can include any configuration where the first, undeployed, profile is smaller than the second, deployed, profile.

The notches 116, 118 can, for example, be used to control the shape and height of the device during deployment. In operation, the notches act like hinges that act to control the device expansion. As will be appreciated further below, when the device is expanded, the two upper edges 120, 120′ of the notch closes in on itself. When the notch edges 120, 120′ abut one another, expansion stops. In this embodiment, the notches occur in opposing pairs along the length of a slit and the notches are positioned symmetrically along the length of the slit.

The strut portion of the elongate expandable shaft 110 is the section of the elongate shaft that is positioned between the slits. Where, for example, where there are four slits, as depicted in this embodiment, there are four struts, each strut defining an edge along a long axis of the slit. The strut 122 has a leading exterior surface that forms a cutting surface 126 adapted to cut through cancellous bone. As the cutting surface 126 abuts the harder cortical bone that forms the exterior shell of the bony structure, the leading cutting surface merges into a cortical bone support surface 128. This can occur by, for example, the surface flattening out as it applies force to the harder interior cortical bone surface. Once in place, the strut provides a structural member that sustains an axial compressive load to the device.

Turning now to FIG. 2B, the device 100 is depicted from a side view. In this depiction, the device 100 has two pairs of opposing slits 114, 115. Where four slits are employed, the each slit of a pair of slits is positioned at 180° angle from the other slit. Thus, when looking at a device 100 from a side view where the device lies flat within a plane, a pair of opposing slits is positioned so that the view is through the first slit and then through the opposing second slit as depicted in FIG. 2B. However, as will be appreciated by those skilled in the art, in addition to employing a configuration with pairs of opposing slits, a configuration with, for example, three slits could be used without departing from the scope of the invention. Where three slits are employed, the slits would occur at 120° intervals around the 360° circumference of the device.

Lines C-C, D-D and E-E, shown in FIG. 2B correspond to the cross-sectional views FIGS. 2C-E, which are taken along the lines C-C, D-D and E-E with the view down the length of the device in the direction of the arrows. As appreciated from FIG. 2C, along at least a portion of its length, the device 100 has a continuous circumference 130 consistent with an elongate expandable shaft 110 with a hollow lumen 112. Where cross-sectional view is taken across a portion of device 100 where the slit 114 is, the device has, for example, four solid sections 132, 132′ that correspond to the expandable sections of the device and form the struts 122. Each slit 114, 114′ in this Figure appears as a channel 134, 134′ extending into the page in the direction of the arrows C-C shown in FIG. 2B. Where the device is cross-cut along a section of the device corresponding to the notch, a portion appears as a channel 134, 134′ with a shorter, widened section 136, 136′. As will be appreciated by those skilled in the art, the device can be configured to have two or more slits, forming two or more struts. Further, the cross-sectional shape of the device can be circular, as depicted, oval, elliptical, or any other shape suitable to achieve the results desired.

From an end view depicted in FIG. 2C, a cross-section taken along the lines F-F is depicted in FIG. 2F. The cross-section is taken through the elongate expandable shaft 110, slits 114, 114′ and notches 116, 118.

Turning now to FIGS. 2G-I various view of the device are depicted in an expanding condition having a second, expanded, profile 111′. The second, expanded, profile 111′ as depicted by changes in diameter, d, d1, d2 along its length. As the device 100 expands, the sections of the device that form around the slits, e.g. struts, extend radially away from the central lumen c of the device. However, as described above, the second, expanded, profile 111′ can differ from the first profile by merely being larger or different in diameter or circumference and need not be distinguished by having a variable diameter or circumference along its length. As the space between the walls of the slits 134, 134′ increases, the edges of the notches 120, 120′ approach each other. Movement of the walls of the slits away from each other stops when the edges of the notches abut. As illustrated in FIGS. 2K-L the space between the walls of the slits increases (i.e., the distance between the struts increases) along its length, and increases as the device is deployed. Concurrently, the length of the device can decrease during the process. FIG. 2M illustrates a cross-sectional view of the device in a deployed, or partially deployed, condition. As will be appreciated by those of skill in the art, the walls of the notches are configured to prevent further expansion of the device. However, the walls of the notches need not reach the stopping point in order for the device to be deployed.

FIGS. 2N-Q illustrate the process of deploying the device 100 to treat cortical bone. For purposes of illustration, the device is depicted deployed within a vertebral body 14 having a fracture 80. However, as will be appreciated by those skilled in the art, the device can be deployed in any target bone structure. The device is particularly adapted for use with cancellous bone, which has a porosity of 30-90%. The device 100 is inserted into the vertebral body using a delivery device 150, in this case at an angle that does not correspond to an axial plane 52. Once the device 100 is far enough into the target space, it is deployed as illustrated in FIG. 2Q. As described above, the distal end 138′ of the device 100 can be positioned entirely within the cancellous bone 42 or at least partially within the cortical bone 40. Deploying the device 100 enables the struts 122 to cut through the cancellous bone 42 until each strut 122, 122′ abuts the cortical bone 40. The struts apply a force of, for example, 2 psi to over 100 psi to cut through the cancellous bone, depending upon the porosity of the cancellous bone and the anatomical location. Once the strut abuts the cortical bone it ceases cutting through bone because the force applied by the device is sufficient to cut through cancellous bone but insufficient to cut completely through the cortical bone. Thus the force applied is a stabilization force which is applied to the surface of the cortical bone in an amount sufficient to stabilize the cortical bone or lift opposing cortical bone surfaces away from each other and restore, or substantially restore, the distance or height h between the cortical bone surfaces. Once the distance has been restored, or substantially restored, as shown in FIG. 2R, material 142 can be injected via the delivery device into the space 46 formed between the cortical bone surfaces. Thereafter, as depicted in FIG. 2S, the deployed device 100 can be detached from the delivery device and left within the bone. Where the material 142 is injected through the device 100, the device 100 operates as a cannula, or tube optionally fitted with a trocar, that is used to inject material into the bone.

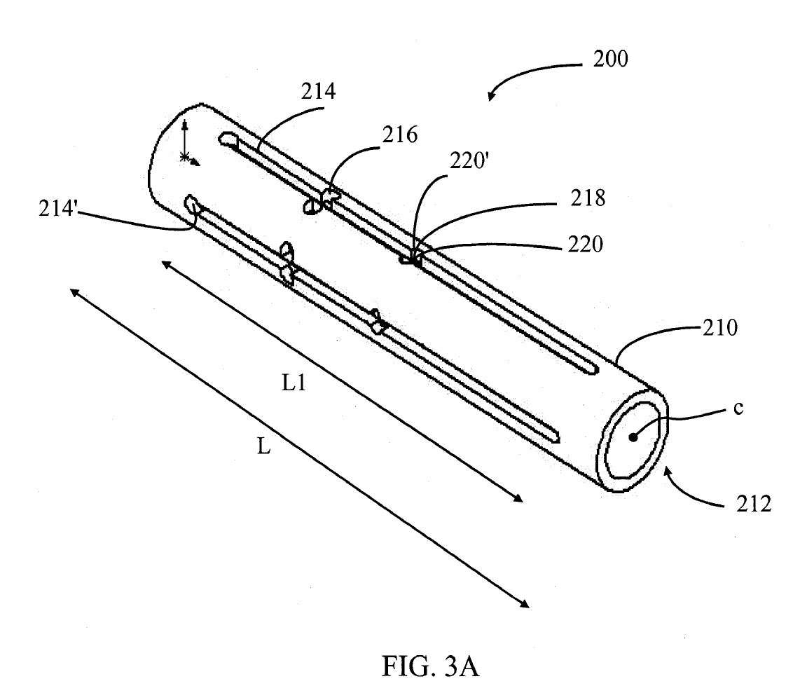

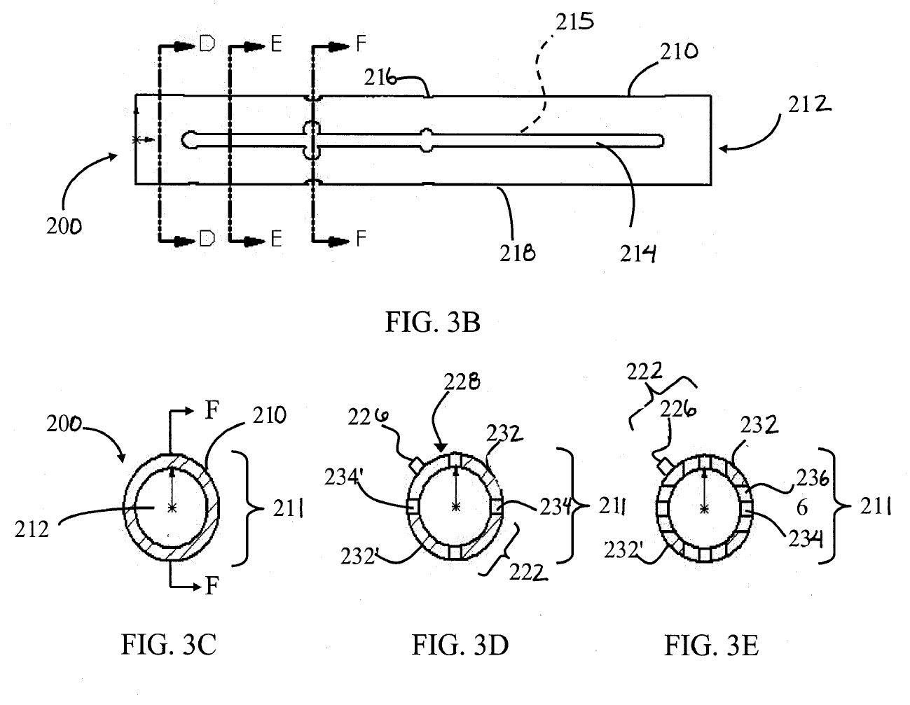

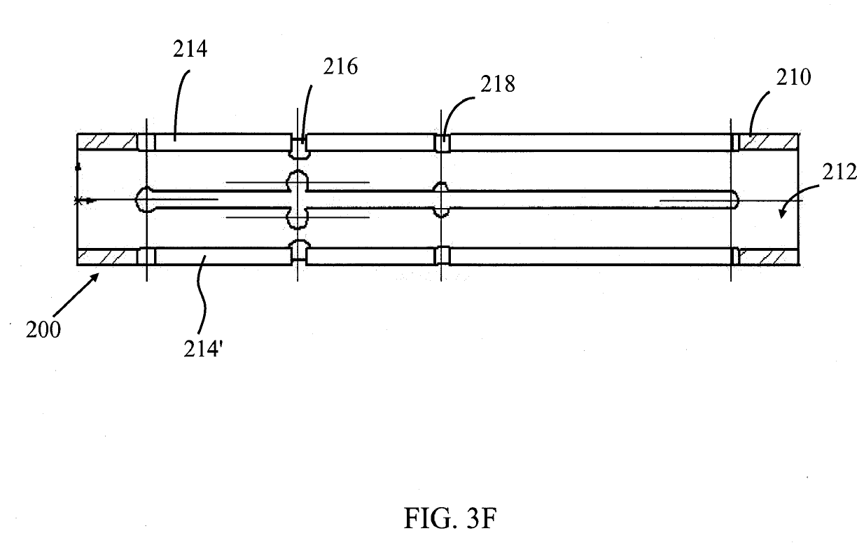

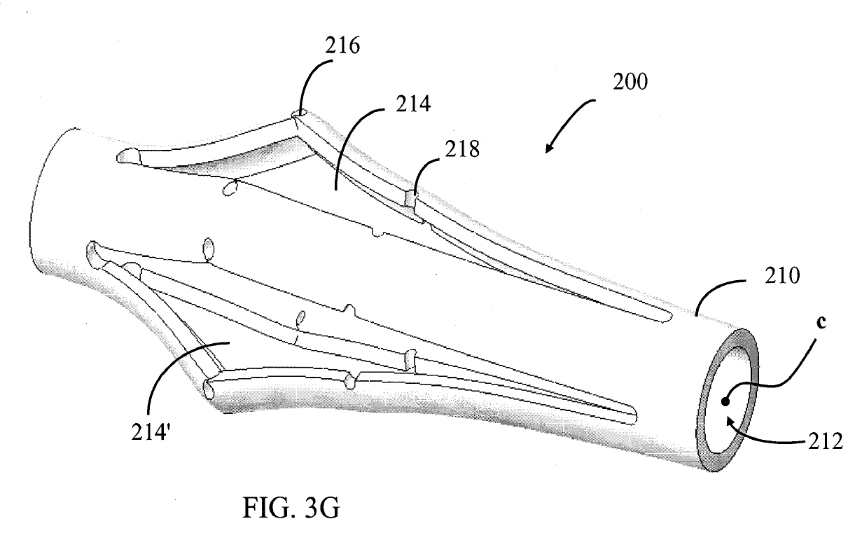

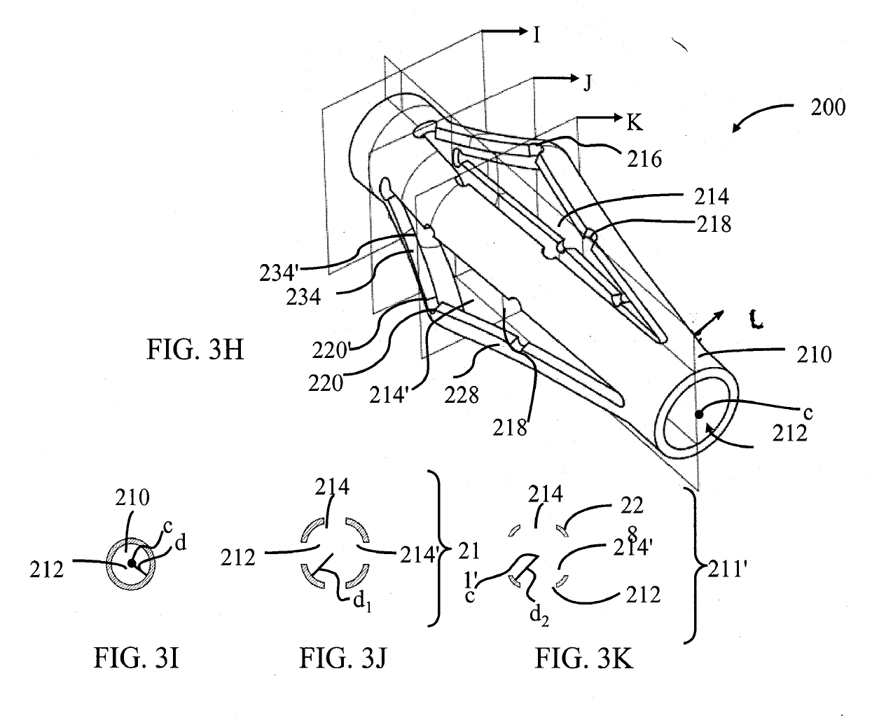

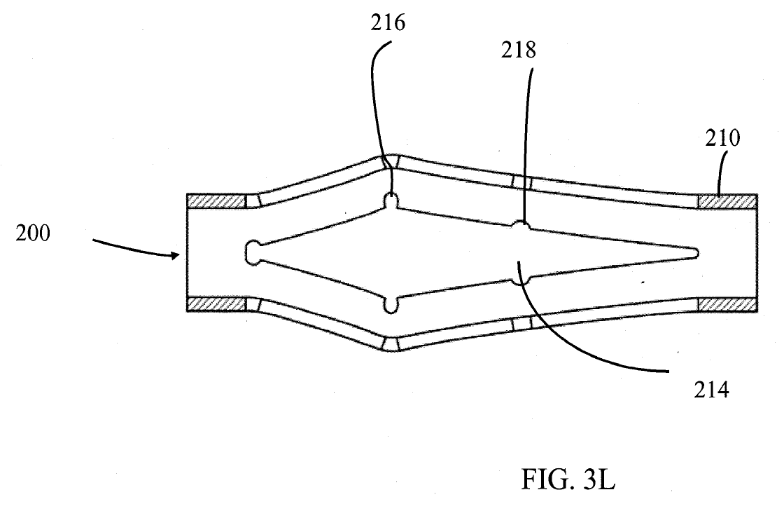

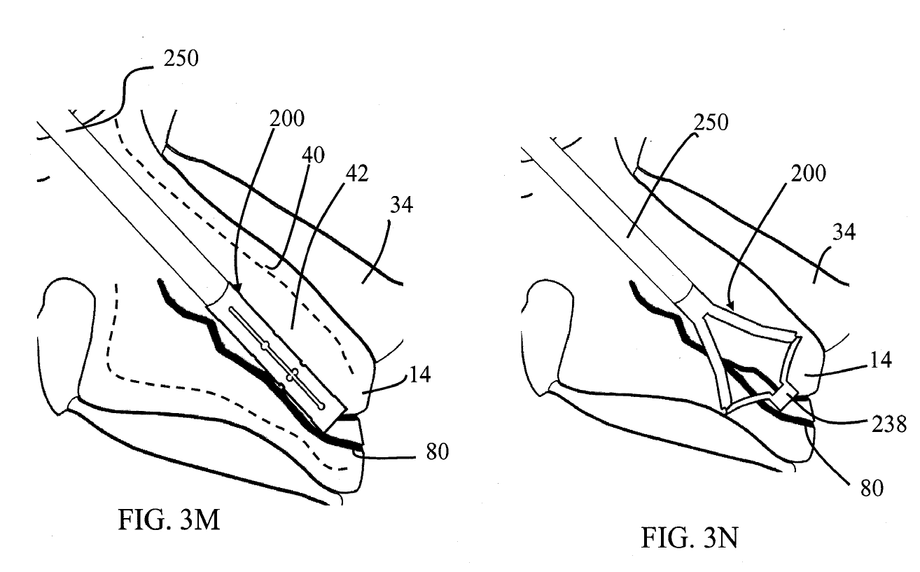

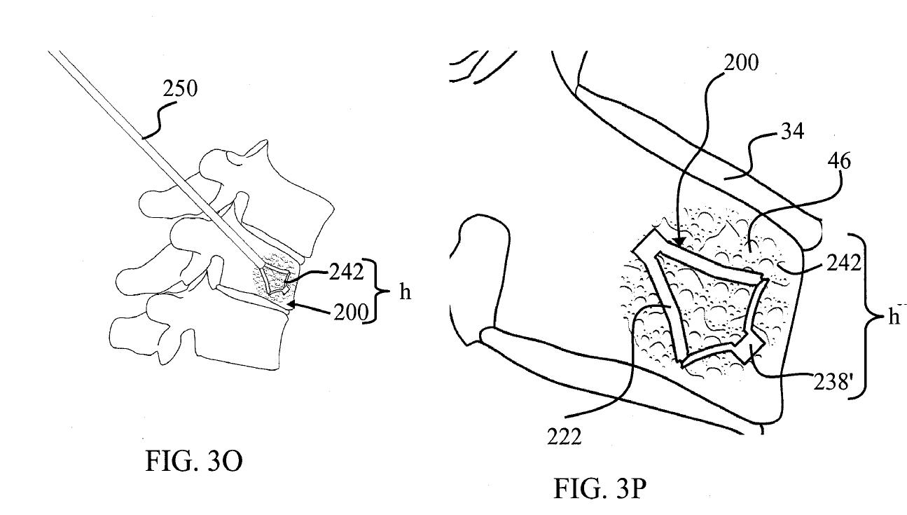

FIG. 3A illustrates an alternative embodiment of a perspective view of an expandable stabilization device 200 suitable for use completely or partially within a vertebral body 14. The expandable device 200 has an elongate expandable shaft 210 adapted to be positioned within cancellous bone 42, such as the cancellous bone 42 of a vertebral body 14. The non-expandable ends of the device can also be positioned within the cancellous bone, or one end can be partially positioned within cortical bone in order to secure the position of the device.

The elongate expandable shaft 210, as depicted, has a hollow central lumen 212 and two or more slits 214, 214′ along at least a portion L₁ of the length L of the shaft 210. Each slit 214 in this embodiment is configured to have one or more notches 216, 218 which operate substantially as described above with respect to FIG. 2. As provided for in this configuration, the notches are positioned asymmetrically along the length of the device.

The strut portion of the elongate expandable shaft 210 is the section of the elongate shaft that is positioned between the slits. Where, for example, there are four slits, as depicted in this embodiment, there are four struts. The strut 222 has a leading exterior surface that forms a cutting surface 226 adapted to cut through cortical bone. As the cutting surface 226 abuts harder cortical bone, the leading cutting surface merges into a cortical bone support surface 228. Once in place, the strut provides a structural member that sustains an axial compressive load to the device.

Turning now to FIG. 3B, the device 200 is depicted from a side view. In this depiction, the device 200 has two pairs of opposing slits 214, 215. Similar to the embodiment described above, where four slits are employed, each slit of a pair of slits is positioned at 180° angle from the other slit. Thus, when looking at a device 200 from a side view where the device lies flat within a plane, a pair of opposing slits is positioned so that the view is through the first slit and then through the opposing second slit as depicted in FIG. 3B. However, as will be appreciated by those skilled in the art, in addition to employing a configuration with pairs of opposing slits, a configuration with, for example, three slits could be used without departing from the scope of the invention. Where three slits are employed, the slits would occur at 120° intervals around the 360° circumference of the device.

Lines C-C, D-D and E-E, shown in FIG. 3B correspond to the cross-sectional views FIGS. 3C-E, which are taken along the lines C-C, D-D and E-E with the view down the length of the device in the direction of the arrows. As appreciated from FIG. 3C, along at least a portion of its length, the device 200 has a continuous circumference 230 consistent with an elongate expandable shaft 210 with a hollow lumen 212. Where cross-sectional view is taken across a portion of device 200 where the slit 214 is, the device has, for example, four solid sections 232, 232′ that correspond to the expandable sections of the device and form the struts 222. Each slit 214, 214′ in this Figure appears as a channel 234, 234′ extending into the page in the direction of the arrows C-C shown in FIG. 3B. Where the device is cross-cut along a section of the device corresponding to the notch, a portion appears as a channel 234, 234′ with a shorter, widened section 236, 236′. As will be appreciated by those skilled in the art, the device can be configured to have two or more slits, forming two or more struts. Further, the cross-sectional shape of the device can be circular, as depicted, oval, elliptical, or any other shape suitable to achieve the results desired without departing from the scope of the invention.

From an end view depicted in FIG. 3C, a cross-section taken along the lines F-F is depicted in FIG. 3F. The cross-section is taken through the elongate expandable shaft 210, slits 214, 214′and notches 216, 218.

Turning now to FIGS. 3G-L various view of the device are depicted in an expanding condition having a second, expanded, profile 211′. The second, expanded, profile 211′ as depicted by changes in diameter, d, d1, d2 along its length. Due to the fact that the notches 216, 218 are positioned asymmetrically along the length of the device, the device 200 the device profile at d2 will be the highest along the length and will be positioned along the length L2. As the device 200 expands, the sections of the device that form around the slits extend radially away from the central lumen c of the device. However, as described above, the second, expanded, profile 211′ can differ from the first profile by merely being larger or different in diameter and need not be distinguished by having a variable diameter. As the space between the walls of the slits 234, 234′ increases, the upper edges of the notches 220, 220′ approach each other. Movement of the walls of the slits away from each other stops when the edges of the notches abut. As illustrated in FIGS. 3I-K the space between the slits increases along its length, and increases as the device is deployed. FIG. 3L illustrates a cross-sectional view of the device in a deployed, or partially deployed, condition. As will be appreciated by those of skill in the art, the walls of the notches are configured to prevent further expansion of the device. However, the walls of the notches need not reach the stopping point in order for the device to be deployed.

FIGS. 3M-P illustrate the process of deploying the device 200 to treat bone. For purposes of illustration, the device is depicted deployed within a vertebral body 14 having a fracture 80. The device 200 is inserted into the vertebral body using a delivery device 250, in this case at an angle that does not correspond to an axial plane 52. Once the device 200 is far enough into the target space, it is deployed as illustrated in FIG. 3N. As described above, the distal end 238′ of the device 200 can be positioned entirely within the cancellous bone 42 or at least partially within the cortical bone 40. Once deployed, the highest profile of the device is positioned distally 238′ along its length, in order to facilitate providing separation to the cortical bone surfaces at an end of the vertebral body furthest away from the proximal 238 entry site of the device. This configuration is particularly suitable where the vertebral body has lost height along one side in such a manner that the vertebral body acquires a wedge-like profile. Deploying the device 200 enables the struts 222 to cut through the cancellous bone 42 until each strut 222, 222′ abuts the cortical bone 40. Once the strut abuts the cortical bone it ceases cutting through bone and begins applying force to the surface of the cortical bone in an amount sufficient to lift the opposing cortical bone surfaces away from each other and restore, or substantially restore, the distance or height h between the cortical bone surfaces. This restoration restores, or substantially restores the original profile, that has been altered as a result of micro-architecture deterioration of bone tissue. Once the distance has been restored, or substantially restored, bone has been achieved as shown in FIG. 3O, material 242 can be injected via the delivery device into the space 46 formed between the cortical bone surfaces. Thereafter, as depicted in FIG. 3P, the deployed device 200 can be detached from the delivery device and left within the bone.

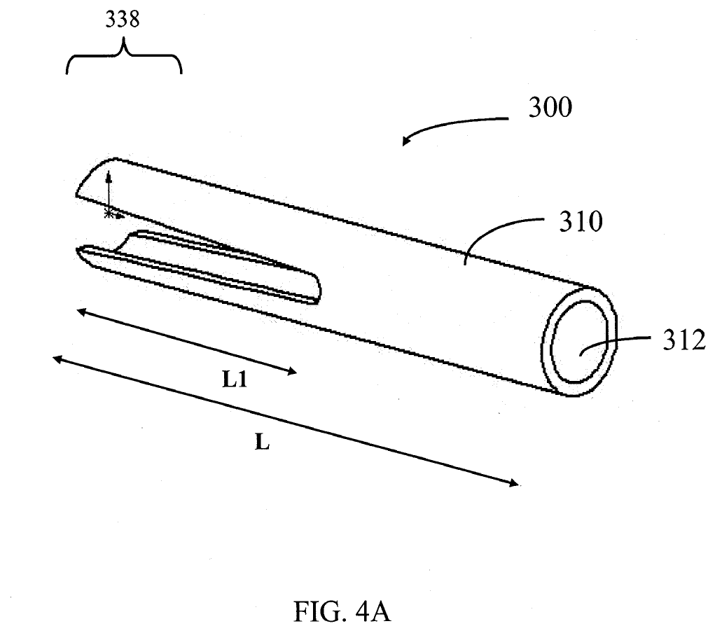

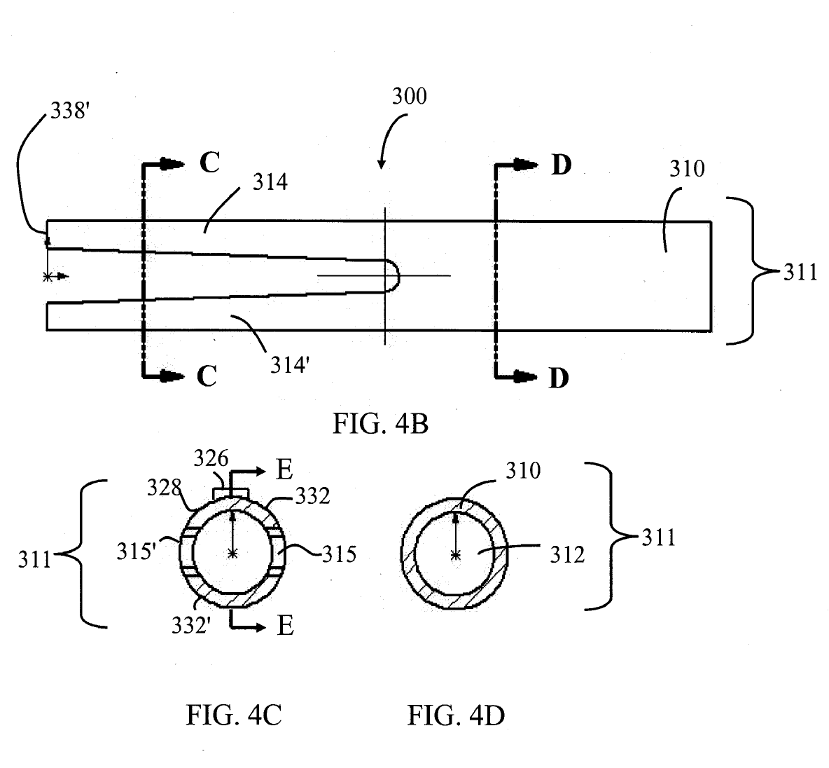

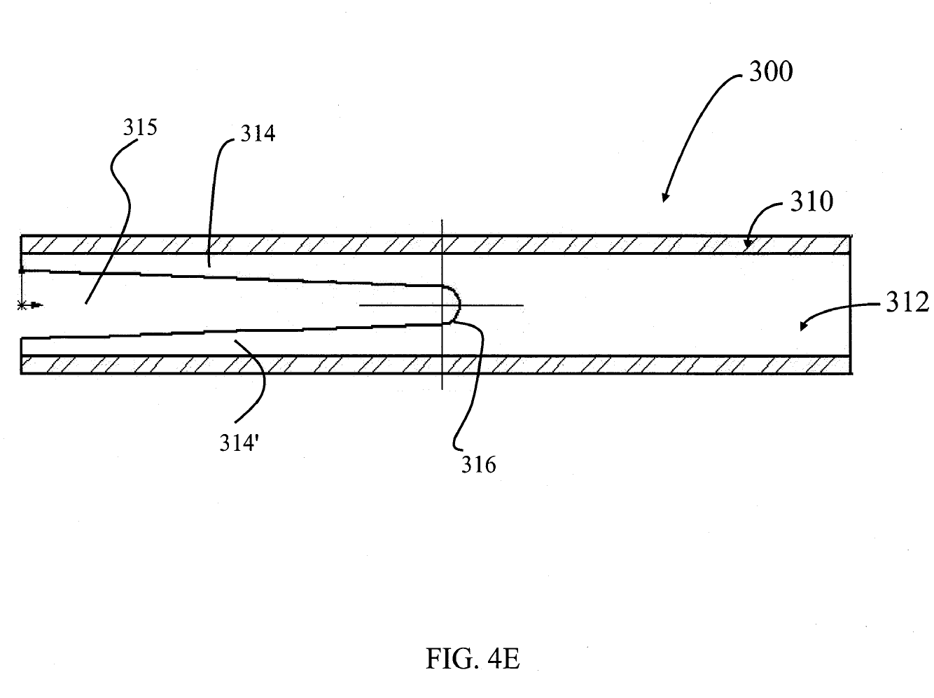

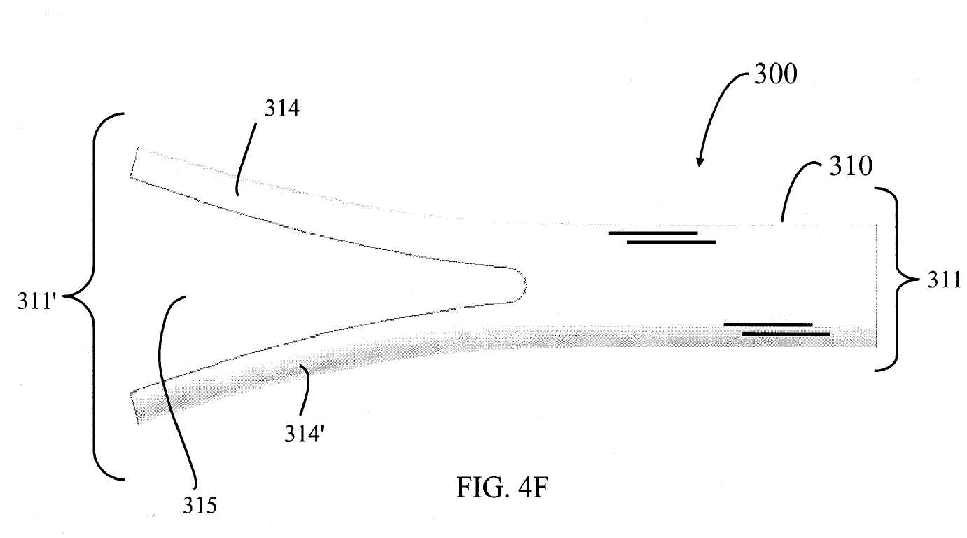

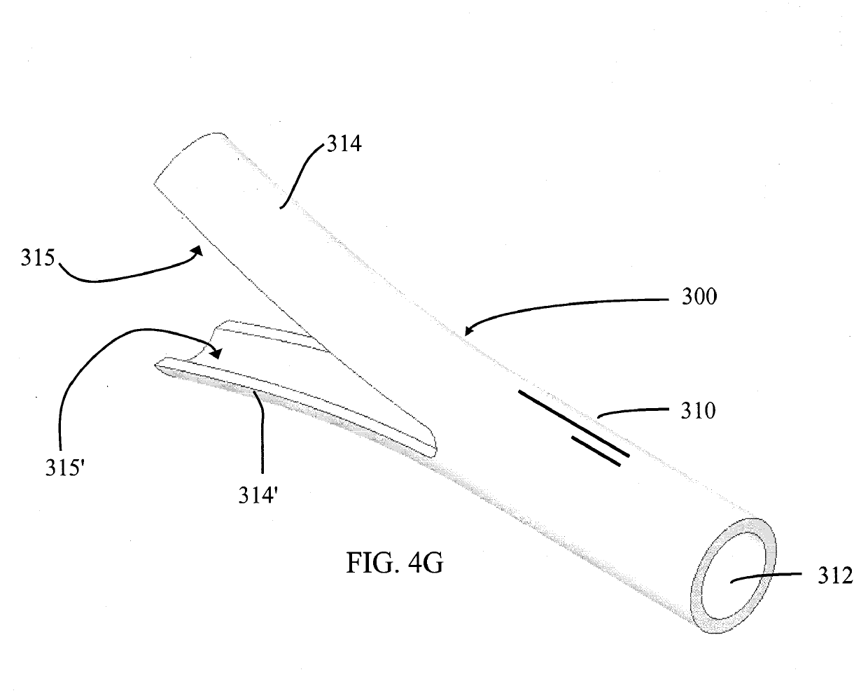

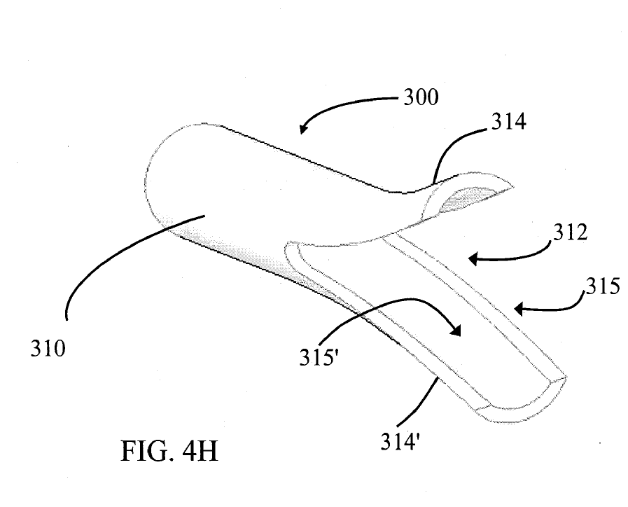

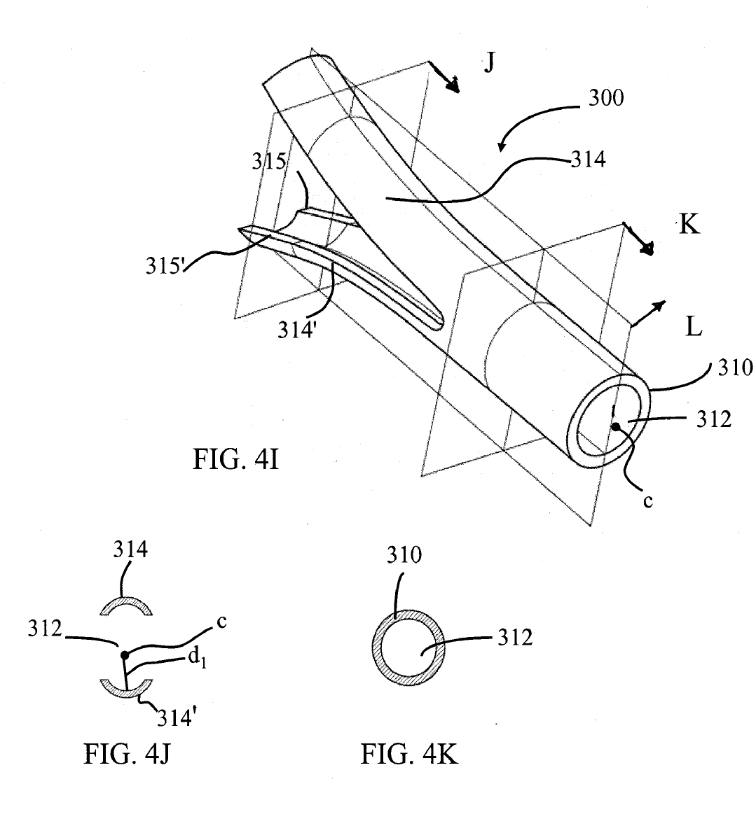

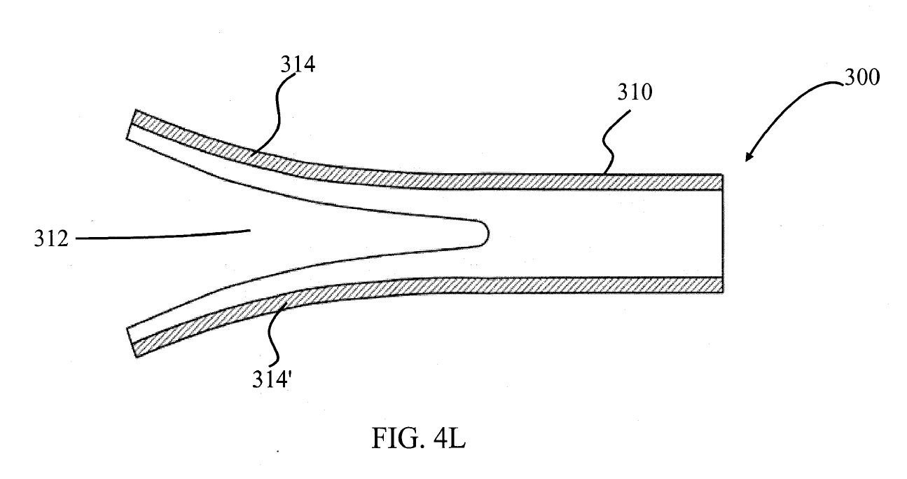

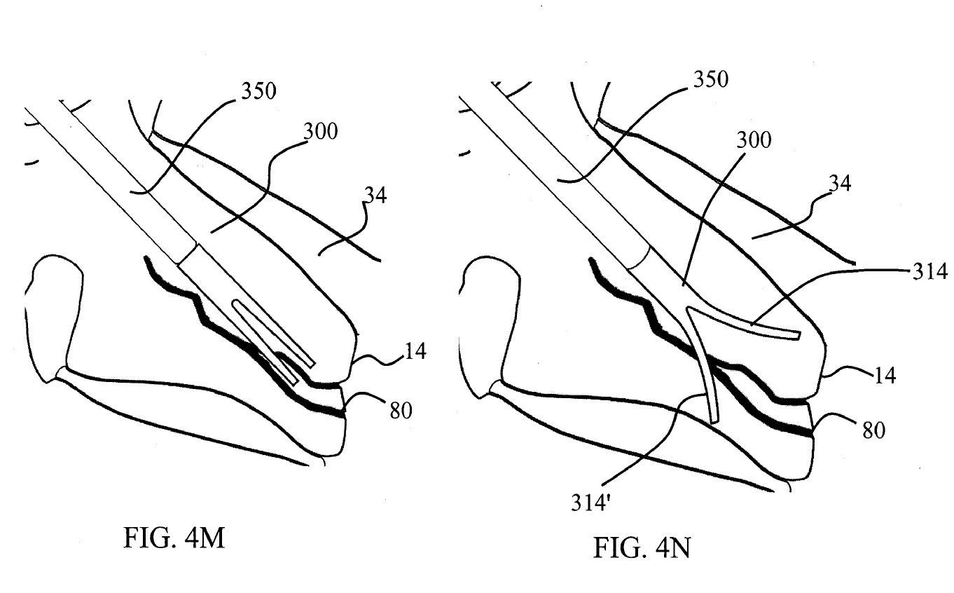

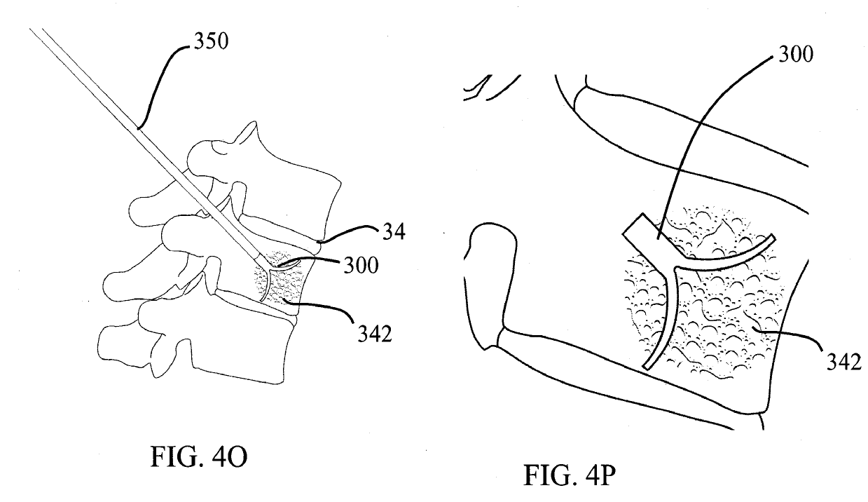

FIG. 4A illustrates a perspective view of yet another expandable stabilization device 300 suitable for use completely or partially within, for example, a vertebral body 14. The expandable device 300 has an elongate expandable shaft 310 adapted to be positioned within cancellous bone 42, such as the cancellous bone 42 of a vertebral body 14.

The elongate expandable shaft 310, as depicted, has a hollow central lumen 312 and two or more arms 314, 314′ formed along at least a portion L₁ of the length L of the shaft 310 at its distal end 338′. Each slit forming the arm 314 can also be configured to have a notch 316 at the proximal end of the slit.

The device depicted in FIGS. 4B-D is in a first undeployed profile 311. As depicted the undeployed profile 311 has a constant circumference along its length. However, as discussed above with respect to other embodiments, the undeployed profile is not restricted to a device having a constant profile along its length and can include any configuration where the first, undeployed, profile is smaller than the second, deployed, profile.

The strut portion of the elongate expandable shaft 310 in this embodiment is the arm 316. Where, for example, there are four slits, as depicted in this embodiment, there are four struts. The arm 316 has a leading exterior surface that forms a cutting surface 326 adapted to cut through cortical bone. As the cutting surface 326 abuts harder cortical bone, the leading cutting surface merges into a cortical bone support surface 328. Once in place, the struts or arms provide a structural member that sustains an axial compressive load to the device.

Turning now to FIG. 4B, the device 300 is depicted from a side view. In this depiction, the device 300 has two pairs of opposing slits 315, 315′. Thus, when looking at a device 300 from a side view where the device lies flat within a plane, a pair of opposing slits is positioned so that the view is through the first slit and then through the opposing second slit as depicted in FIG. 4B. However, as will be appreciated by those skilled in the art, in addition to employing a configuration with one or more pairs of opposing slits, a configuration with, for example, three slits, or multiples thereof, could be used without departing from the scope of the invention. Where three slits are employed, the slits would occur at 120° intervals around the 360° circumference of the device.

Lines C-C, and D-D shown in FIG. 4B correspond to the cross-sectional views FIGS. 4C-D, which are taken along the lines C-C, and D-D with the view down the length of the device in the direction of the arrows. As appreciated from FIG. 4C, along at least a portion of its length, the device 300 has a continuous circumference 330 consistent with an elongate expandable shaft 310 with a hollow lumen 312. Where cross-sectional view is taken across a portion of device 300 where the slit 315 is, the device has, for example, two solid sections 332, 332′ that correspond to the expandable sections of the device and form the arms or struts 322. Each slit 314, 314′ in this Figure appears as a channel 334, 334′ extending into the page in the direction of the arrows C-C shown in FIG. 4B. As will be appreciated by those skilled in the art, the device can be configured to have two or more slits, forming two or more struts or arms. Further, the cross-sectional shape of the device can be circular, as depicted, oval, elliptical, or any other shape suitable to achieve the results desired.

From an end view depicted in FIG. 4C, a cross-section taken along the lines E-E is depicted in FIG. 4E. The cross-section is taken through the elongate expandable shaft 310, slits 315, 315′.

Turning now to FIGS. 4F-I various view of the device are depicted in an expanding condition having a second, expanded, profile 311′. The second, expanded, profile 311′ as depicted by changes in diameter, d, d1, d2 along its length. As the device 300 expands, the sections of the device that form around the slits extend radially away from the central lumen c of the device. However, as described above, the second, expanded, profile 311′ can differ from the first profile by merely being larger or different in diameter and need not be distinguished by having a variable diameter. As illustrated in FIGS. 4J-K the space between the slits increases along its length, and increases as the device is deployed. FIG. 4L illustrates a cross-sectional view of the device in a deployed, or partially deployed, condition.

FIGS. 4M-P illustrate the process of deploying the device 300 to treat bone. For purposes of illustration, the device is depicted deployed within a vertebral body 14 having a fracture 80. The device 300 is inserted into the vertebral body using a delivery device 350, in this case at an angle that does not correspond to an axial plane 52. Once the device 300 is far enough into the target space, it is deployed as illustrated in FIG. 4N. In this embodiment, the distal end 338′ of the device 300 is the portion of the device that expands to support the cortical bone. Therefore, in this embodiment the distal end 338′ is not positioned within the cortical bone 40. Deploying the device 300 enables the struts 322 to cut through the cancellous bone 42 until each strut 322, 322′ abuts the cortical bone 40. Once the strut abuts the cortical bone it ceases cutting through bone and begins applying force to the surface of the cortical bone in an amount sufficient to lift the opposing cortical bone surfaces away from each other and restore, or substantially restore, the distance or height h between the cortical bone surfaces. Once the distance has been restored, or substantially restored, as shown in FIG. 4O, material 342 can be injected vial the delivery device into the space 46 formed between the cortical bone surfaces. Thereafter, as depicted in FIG. 4P, the deployed device 300 can be detached from the deliver device and left within the bone.

The embodiments shown in FIGS. 2-4 enable the user to achieve separation of two cortical bone surfaces at variable positions along the length of the device. The configuration of FIG. 2, with its symmetrical configuration of notches along the length of slits, allows for the device to deploy within the cancellous space providing an even force to the cutting surface and an even force to the support structure. The configuration of FIG. 3, with its asymmetrical configuration of notches along the length of slits, allows for the device to deploy within the cancellous space providing a greater force asymmetrically to the cutting surface and a greater force asymmetrically to the support structure. The configuration of FIG. 4, with its open strut configuration, allows for the device to deploy within the cancellous space providing a greater amount of force to the cutting surface at the distal end and a greater amount of force to the support structure at the distal end.

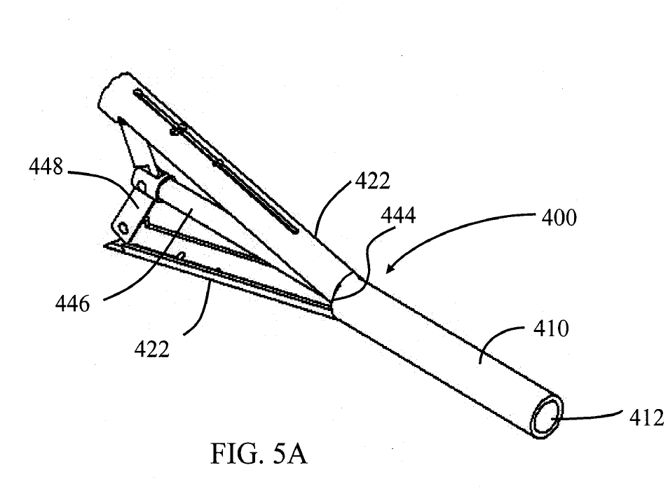

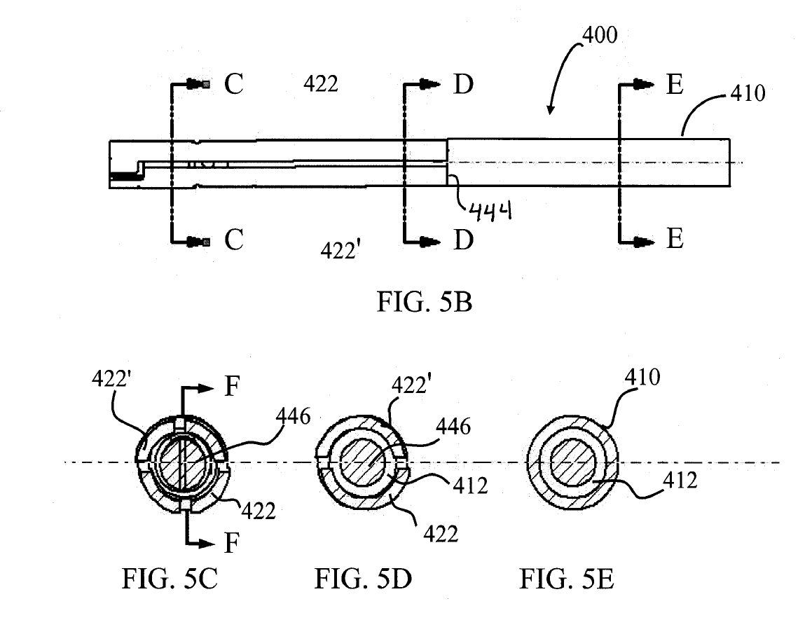

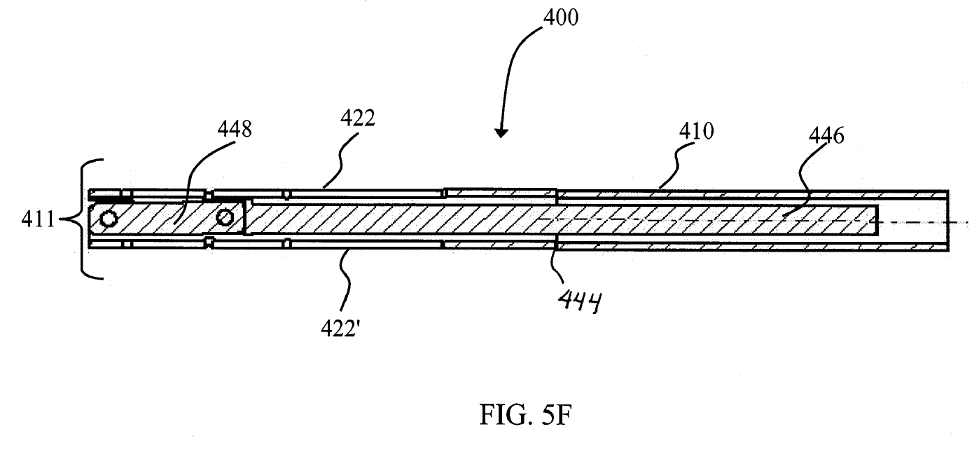

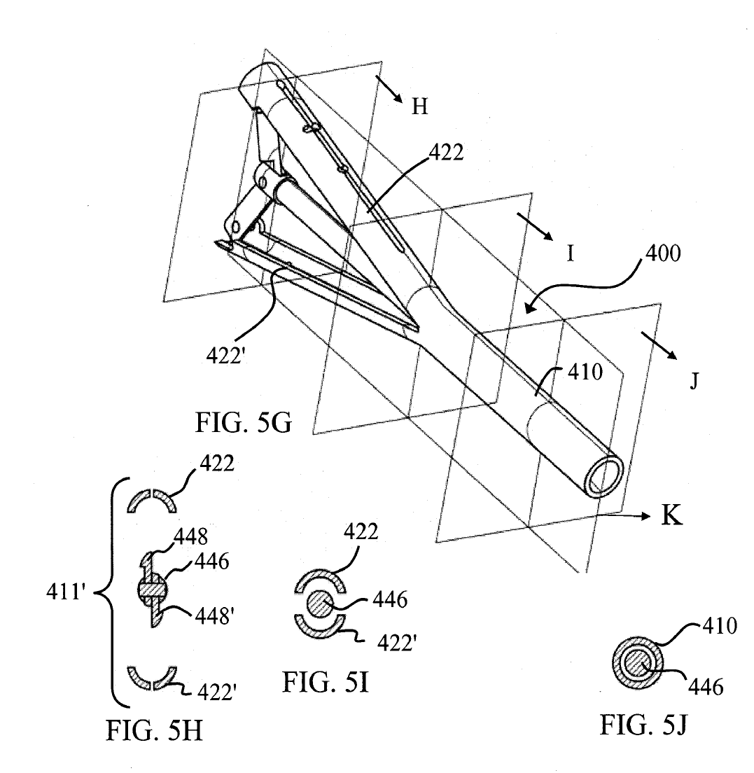

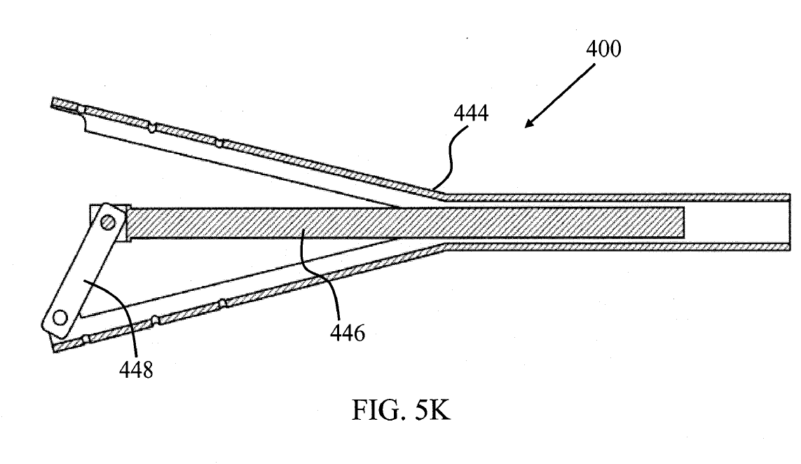

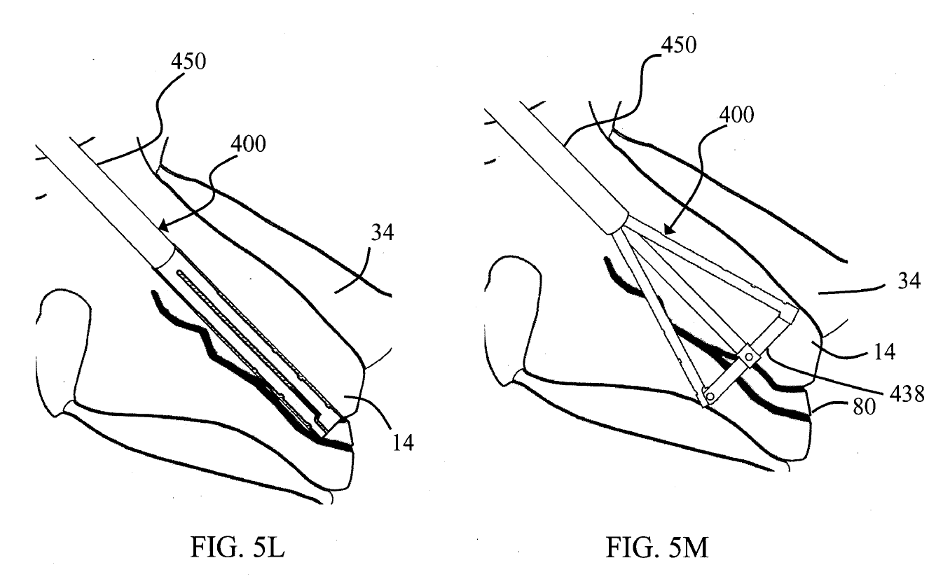

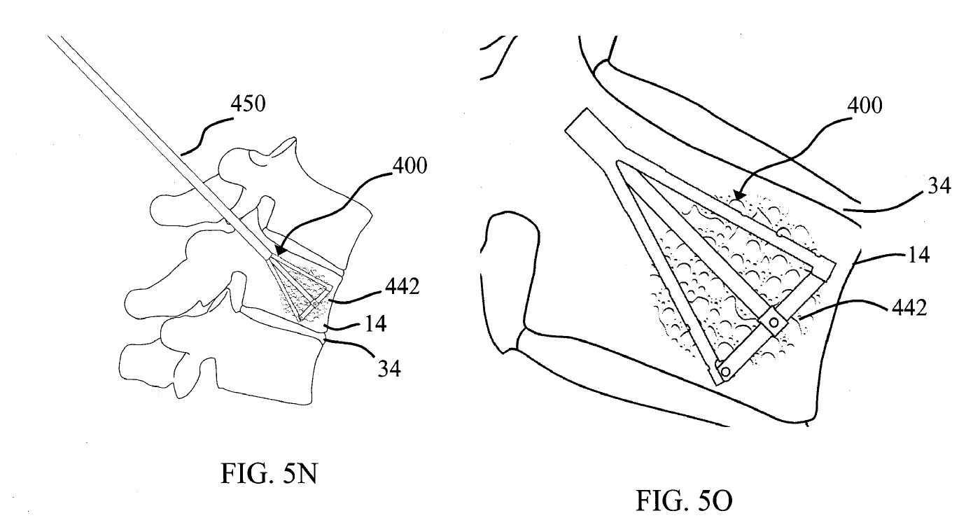

Turnng now to FIG. 5, a device 400 is depicted having an elongate shaft 410 that opens into a deployed condition along its distal end. Arms 422 are provided that are either formed integrally within the elongate shaft 410 or are adapted to engage the elongate shaft. The arms 422 are configured such that the arms can be moved away from a central lumen of the device by pivoting the arms open and/or pivoting the arms across a joint 444. Activation of the arms can be achieved by providing a control member, such as rod 446 that can be advanced within the central lumen 412 of the device. As the rod is moved distally, the rod engages support beams 448 that are positioned in series with the activation rod and then move into a position vertical to the rod at activation supporting the arms 442 in an open configuration. The beams can be notched in order to lock into place upon activation, if desired. As with previous embodiments, material 442 can be injected that further supports the device in place. Once activated, the arms 422 extend away from a central lumen c to cut through cancellous bone. Once the arms extend out to a desired position, the arms adapt to cortical bone support members.

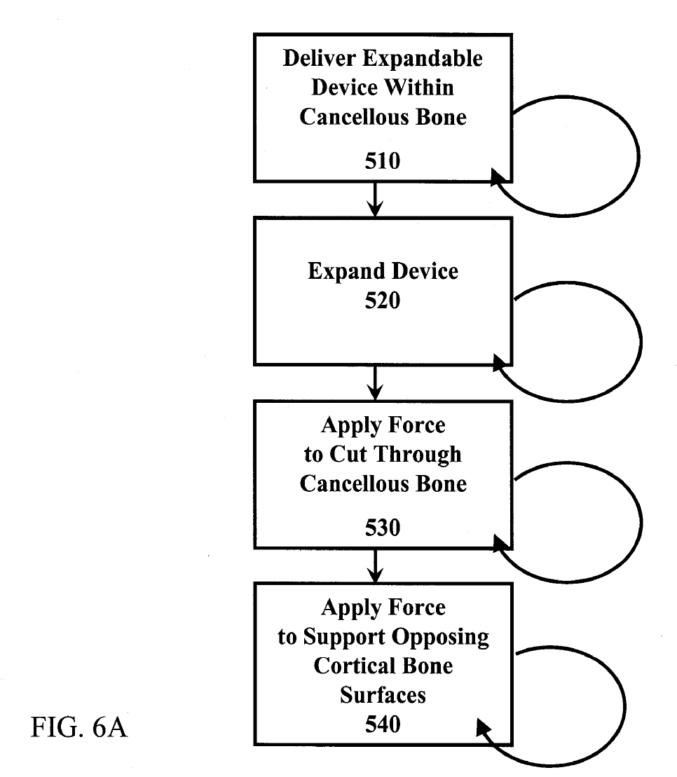

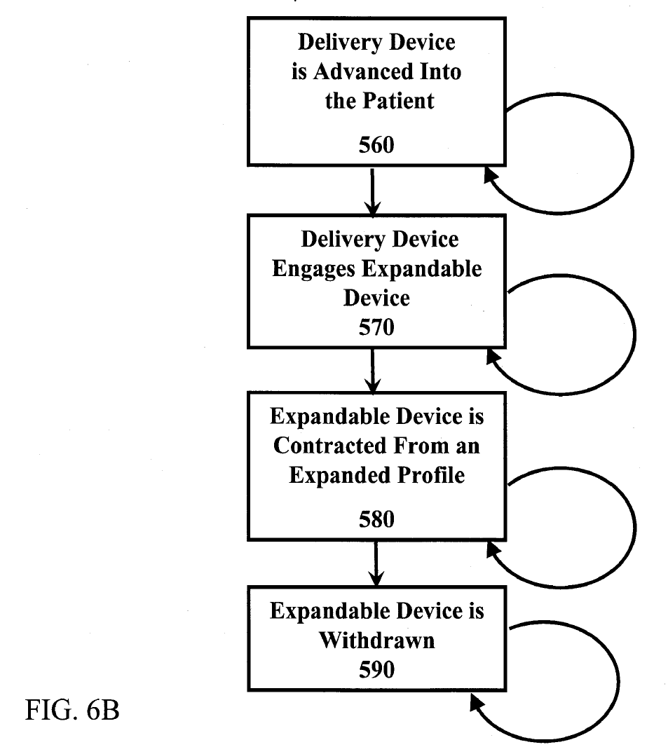

FIG. 6A illustrates the steps of a method for deploying a device of the invention, such as those detailed above with respect to FIGS. 2-5. In performing the method of the invention, a device is delivered within the cancellous bone 510. This step can be performed after the step of making a pilot access hole. Alternatively, depending upon the configuration of the device, the device can be configured to provide the access hole and position the device in one step. This step can be repeated one or more times. For example, where an initial device is delivered and a physician, or other user, decides to replace the initial device with a different device, the initially delivered device can be removed and replaced with a new device. Once the device is delivered within the cancellous bone it can optionally be positioned so that a portion of its distal end engages a portion of cortical bone. For example, a portion of the device could be positioned to fit within an aperture created on an interior surface of the cortical bone to anchor the device in place. Once the device is positioned in a desired location, the device is expanded 520. Expansion of the device applies a cutting force from a cutting surface of the device through the cancellous bone 530. The force applied can be any force suitable to cut through cancellous bone, for example, 2 psi to greater than 100 psi. However, the amount of force required will vary depending upon the porosity of the bone, which can range from 30-90%, as well as the anatomical location.

Once the device cuts through the cancellous bone and reaches opposing cortical bone surfaces, the support surface of the device applies a force to the opposing cortical bone surfaces 540 sufficient to either stabilize the position of the position of the opposing cortical bone surfaces or to create a space or gap between the cortical bone surfaces. Creating the space or gap serves to restore the position of the cortical bone surfaces relative to one another. Optionally, a material, such as PMMA, can be introduced through the device into the space between the cortical bone surfaces. A variety of materials are suitable including Once the device is positioned at a desired location, the delivery device is withdrawn, leaving the device positioned within the cancellous space.

FIG. 6B illustrates the steps of a method for removing an implanted device, such as the devices detailed above with respect to FIGS. 2-5. In performing the method of removal the invention, a delivery device is advanced into a cancellous bone 560 to engage the device 570. Once the delivery device engages the implanted device, the device is contracted 580 to reduce the profile of the device from a deployed profile to a non-deployed profile, or substantially to a non-deployed profile. Once the profile of the device is sufficiently reduced, it is withdrawn 590 from the interior of the bone.

As will be appreciated by those skilled in the art, the size of the devices disclosed herein will vary depending upon the target location for treatment. Where the devices are deployed within a vertebral body, the elongate shaft can be configured to have an undeployed diameter of from 2 mm to 10 mm and a deployed diameter of from 6 mm to 35 mm, along at least a portion of its length. The devices can typically have an undeployed length of from, for example, 8 mm to 60 mm. As the devices are deployed, the length of the devices will shorten as the struts expand radially away from an initial configuration and away from the central lumen of the device.

Additionally, the devices can be configured such that the exterior surface of all, or a part, of the device is textured. Texturing can be employed where, for example, it is desirable to prevent movement or slippage of the device in situ. Texturing includes, but is not limited to, dimples, nubs, knurls, teeth, etc.

In some embodiments of the devices disclosed above, an additional controller is provided to control the expansion of the device upon deployment. The controller can be a ratchet, a self-expanding wire, a push control, screw-type, retracting sheath, or any other suitable mechanism adapted to facilitate controlled delivery of the device.

Materials suitable for making the tools and devices described herein would be apparent to those of skill in the art and include, but is not limited to biocompatible metals (such as cobalt chromium steel, surgical steels, titanium, titanium alloys, tantalum, tantalum alloys, aluminum, etc.), ceramics, polyethylene, biocompatible polymers, and other materials known in the orthopedic arts. Furthermore, where the devices have bearing surfaces (i.e. surfaces that contact another surface), the surfaces may be formed from biocompatible metals such as cobalt chromium steel, surgical steel, titanium, titanium alloys (such as the nickel titanium alloy Nitinol), tantalum, tantalum alloys, aluminum, etc. Shape memory alloys, such as Nitinol, can also be used to facilitate deployment of the struts of the device to a particular configuration. Other materials might also be employed, such as ceramics, including pyrolytic carbon, and other suitable biocompatible materials known in the art. Portions of the device can also be formed from suitable polymers include polyesters, aromatic esters such as polyalkylene terephthalates, polyamides, polyalkenes, poly(vinyl) fluoride, PTFE, polyarylethyl ketone, and other materials that would be known to those of skill in the art. Various alternative embodiments of the devices and/or components could comprise a flexible polymer section (such as a biocompatible polymer) that is rigidly or semi rigidly fixed.

The device can also be used in combination with, PMMA, bone filler or allograft material. Suitable bone filler material includes, the use of bone material derived from demineralized allogenic or xenogenic bone and can contain substances for example, bone morphogenic protein, which induce bone regeneration at a defect site. Thus a variety of materials are suitable for use as the synthetic, non-biologic or biologic material, including polymers, cement, including cement which comprises in its main phase of microcrystalline magnesium ammonium phosphate, biologically degradable cement, calcium phosphate cements, and any material that is suitable for application in tooth cements, as bone replacement, as bone filler, as bone cement or as bone adhesive. Also included are calcium phosphate cements based on hydroxylapatite (HA) and calcium phosphate cements based on deficient calcium hydroxylapatites (CDHA, calcium deficient hydroxylapatites). See, U.S. Pat. No. 5,405,390 to O'Leary et al. for Osteogenic Composition and Implant Containing Same; U.S. Pat. No. 5,314,476 to Prewett et al. for Demineralized Bone Particles and Flowable Osteogenic Composition Containing Same; U.S. Pat. No. 5,284,655 to Bogdansky et al. for Swollen Demineralized Bone Particles, Flowable Osteogenic Composition Containing Same and Use of the Compositions in the Repair of Osseous Defects; U.S. Pat. No. 5,510,396 to Prewett et al. for Process for Producing Flowable Osteogenic Composition Containing Demineralized Bone Particles; U.S. Pat. No. 4,394,370 to Jeffries for Bone Graft Material for Osseous Defects and Method of Making Same; and U.S. Pat. No. 4,472,840 to Jeffries for Method of Inducing Osseous Formation by Implanting Bone Graft Material, which disclose compositions containing demineralized bone powder. See also U.S. Pat. No. 6,340,477 to Anderson for Bone Matrix Composition and Methods for Making and Using Same, which discloses a bone matrix composition.

In some embodiments, it may be desirable for the device to be fully or partially bioresorbable.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the claims that follow define the scope of the invention and that methods and structures within the scope of the claims and equivalents thereof are covered thereby.

What is claimed is:

1. An expandable stabilization device for deployment within a vertebral body of a spine comprising:
(a) an elongate expandable shaft adapted to be positioned within the vertebral body having a first profile and a second profile;
(b) wherein the shaft is adapted to cut through cancellous bone within the vertebral body during expansion from the first profile to the second profile; and
(c) further wherein the shaft is adapted to abut a surface of cortical bone within the vertebral body without passing therethrough.

2. An expandable stabilization device for deployment within a cancellous bone comprising:
(a) an elongate expandable shaft adapted to be positioned within the cancellous bone having a first profile and a second profile;
(b) wherein the shaft is adapted to cut through cancellous bone during expansion from the first profile to the second profile; and
(c) further wherein the shaft is adapted to abut a surface of cortical bone adjacent the cancellous bone without passing therethrough.

3. The expandable stabilization device of claim 2 wherein the elongate shaft comprises a plurality of surface areas at least a portion of which is a cutting surface adapted to apply a cancellous bone cutting force to the cancellous bone of the vertebral body.

4. The expandable stabilization device of claim 2 wherein the elongate shaft comprises a plurality of cancellous bone cutting surfaces that deliver a force of from 2 psi to 100 psi to the cancellous bone.

5. The expandable stabilization device of claim 2 wherein the elongate shaft has an undeployed diameter of from 2 mm to 10 mm.

6. The expandable stabilization device of claim 2 wherein the elongate shaft has a deployed diameter of from 6 mm to 35 mm along at least a portion of its length.

7. The expandable stabilization device of claim 2 wherein the elongate shaft has a length of from 8 mm to 60 mm.

8. The expandable stabilization device of claim 2 wherein the elongate shaft has 2 or more elongate slits along its length.

9. The expandable stabilization device of claim 8 wherein the elongate slits have asymmetrically positioned notches along its length.

10. The expandable stabilization device of claim 8 wherein the elongate slits have symmetrically positioned notches along its length.

11. The expandable stabilization device of claim 2 wherein the slits are positioned symmetrically or asymmetrically along the length of the shaft.

12. The expandable stabilization device of claim 2 wherein the elongate shaft has a pair of open ended slits at an end of the shaft.

13. The expandable stabilization device of claim 2 wherein the elongate shaft is self-expanding.

14. The expandable stabilization device of claim 2 wherein the elongate shaft is controllably expandable.

15. The expandable stabilization device of claim 2 wherein the elongate shaft is adapted to support a compressive load when expanded.

16. The expandable stabilization device of claim 2 wherein the elongate shaft is adapted to expand to a profile sufficient to achieve a target vertebral body height.

17. The expandable stabilization device of claim 2 wherein the elongate shaft is adapted to expand more in a first dimension than in a second dimension.

18. The expandable stabilization device of claim 2 wherein the elongate shaft is adapted to expand equally in a first dimension and a second dimension.

19. The expandable stabilization device of claim 2 wherein the elongate shaft comprises a first section expandable to a first profile and a second section expandable to a second profile.

20. The expandable stabilization device of claim 2 wherein the device is mounted to a delivery device and adapted to establish a subcutaneous path into the vertebral body.

21. The expandable stabilization device of claim 2 further comprising a control member positioned within a lumen of the shaft configured to expand the shaft from the first profile to the second profile.

22. The expandable stabilization device of claim 2 wherein the device further comprises a lumen through which material is delivered into the vertebral body.

23. The expandable stabilization device of claim 2 wherein the device is formed from a material selected from the group consisting of metals, plastics, composites or memory materials.

24. The expandable stabilization device of claim 2 wherein the device is formed from a biologic or nonbiologic material that promotes fusion.

25. The expandable stabilization device of claim 2 wherein the surface is modified to prevent slippage.

26. The expandable stabilization device of claim 25 wherein the surface is selected from the group consisting of: dimple, nub, knurl, and teeth.

27. The expandable stabilization device of claim 2 wherein the device is formed at least partially from shape memory material.

28. A system for cutting through cancellous bone within a vertebral body of a spine without cutting through cortical bone of the vertebral body comprising:
(a) an expandable body having a first profile and a second profile wherein a surface of the expandable body is adapted to cut through cancellous bone within the vertebral body; and
(b) a delivery device having a distal end adapted to engage the expandable body to deliver the delivery device into the vertebral body.

29. An expandable device adapted to apply force sufficient to cut through cancellous bone within a vertebral body of a spine and insufficient to cut through a cortical bone section of the vertebral body during expansion of the device wherein the device restores a height of a vertebral body to a target height.

30. A method for treating a bone comprising:
(a) delivering an expandable device within a cancellous bone;
(b) expanding the delivered device within the cancellous bone;
(c) applying force from a cutting surface of the device to the cancellous bone sufficient to cut through the cancellous bone; and
(d) applying force from a support surface of the device to an inner surface of a cortical bone sufficient to support the cortical bone.

31. The method of claim 30 further comprising the step of applying force from the surface of the device to the cortical bone of a vertebral body sufficient to increase a distance between two cortical bone surfaces.

32. The method of claim 30 further comprising the step of confirming a position of a vertebral body.

33. The method of claim 30 further comprising the step of administering a material within a space created in a portion of the cancellous bone to facilitate bone restoration.

34. The method of claim 30 further comprising the step of administering a material within a space created in a portion of the cancellous bone to stabilize a position of the device.

35. The method of claim 30 further comprising the step of applying force from the surface of the device to the cortical bone sufficient to increase a distance between a first section of the cortical bone and a second section of the cortical bone at a target location within the bone.

36. The method of claim 30 further comprising the step of applying force from the surface of the device to the cortical bone sufficient to increase a distance between a caudad cortical section of a vertebral body and a cephalad cortical section of a vertebral body.

37. The method of claim 30 further comprising the step of removing a deployed device.

38. The method of claim 37 further comprising the steps of accessing the deployed device; engaging the deployed device with a tool; reducing a profile of the device; and withdrawing the device.

39. A cannula adapted to be deployed within a vertebral body of a spine comprising:
(a) an elongate expandable tube adapted to be positioned within a vertebral body having a first profile and a second profile;
(b) wherein the tube is adapted to cut through cancellous bone located within the vertebral body during expansion from the first profile to the second profile;
(c) further wherein the tube is adapted to deliver a target material through the elongate expandable tube into the vertebral body; and
(d) further wherein the tube is adapted to abut a cortical bone surface within the vertebral body without completely passing therethrough.

40. An expandable device for use in treating a fractured or collapsed vertebral body of a spine comprising:
(a) a device adapted to cut through cancellous bone interior the vertebral body and abut an inner surface of cortical bone of the vertebral body comprising an elongate expandable shaft adapted to be positioned with the vertebral body having a delivery profile and a deployed profile; and
(b) wherein the device selectively expands along its length in the deployed profile to selectively restore the height of a portion of the fractured or collapsed vertebral body to a target height.

41. A system for cutting through cancellous bone of a vertebral body of a spine comprising an expandable body having a selectively expandable surface adapted to expand in situ in an angled direction non-parallel to a median sagittal plane of a body and non-parallel to a transverse plane of a body.

42. A stabilization device for deployment within a vertebral body of a spine comprising:
(a) an elongate expandable shaft having a first profile and a second profile;
(b) a cutting surface on at least a portion of the expandable shaft;
(c) wherein the cutting surface cuts through cancellous bone; and
(d) further wherein the cutting surface abuts a surface of cortical bone within the vertebral body without passing therethrough.
